SEQUENCE LISTING (197 sequences)

SEQ ID NO: 1 - protein, 14 aa, Artificial Sequence
  Synthetic Polypeptide
GSSTGAVTSGNYPN

SEQ ID NO: 2 - protein, 7 aa, Artificial Sequence
  Synthetic Polypeptide
GTKFLAP

SEQ ID NO: 3 - protein, 9 aa, Artificial Sequence
  Synthetic Polypeptide
VLWYSNRWV

SEQ ID NO: 4 - protein, 5 aa, Artificial Sequence
  Synthetic Polypeptide
KYAMN

SEQ ID NO: 5 - protein, 19 aa, Artificial Sequence
  Synthetic Polypeptide
RIRSKYNNYATYYADSVKD

SEQ ID NO: 6 - protein, 14 aa, Artificial Sequence
  Synthetic Polypeptide
HGNFGNSYISYWAY

SEQ ID NO: 7 - protein, 125 aa, Artificial Sequence
  Synthetic Polypeptide
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSS

SEQ ID NO: 8 - protein, 109 aa, Artificial Sequence
  Synthetic Polypeptide
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 9 - protein, 249 aa, Artificial Sequence
  Synthetic Polypeptide
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 10 - protein, 17 aa, Artificial Sequence
  Synthetic Polypeptide
KSSQSVLDSSTNKNSLA

SEQ ID NO: 11 - protein, 7 aa, Artificial Sequence
  Synthetic Polypeptide
WASTRES

SEQ ID NO: 12 - protein, 9 aa, Artificial Sequence
  Synthetic Polypeptide
QQSAHFPIT

SEQ ID NO: 13 - protein, 5 aa, Artificial Sequence
  Synthetic Polypeptide
NYGMN

SEQ ID NO: 14 - protein, 17 aa, Artificial Sequence
  Synthetic Polypeptide
WINTYTGEPTYADKFQG

SEQ ID NO: 15 - protein, 13 aa, Artificial Sequence
  Synthetic Polypeptide
WSWSDGYYVYFDY

SEQ ID NO: 16 - protein, 122 aa, Artificial Sequence
  Synthetic Polypeptide
QVQLVQSGAEVKKPGESVKVSCKASGYTFTNYGMNWVKQAPGQCLEWMGWINTYTGEPTYADKFQGRVTMTTDTSTSTAYMEIRNLGGDDTAVYYCARWSWSDGYYVYFDYWGQGTSVTVSS

SEQ ID NO: 17 - protein, 122 aa, Artificial Sequence
  Synthetic Polypeptide
QVQLVQSGAEVKKPGESVKVSCKASGYTFTNYGMNWVKQAPGQGLEWMGWINTYTGEPTYADKFQGRVTMTTDTSTSTAYMEIRNLGGDDTAVYYCARWSWSDGYYVYFDYWGQGTSVTVSS

SEQ ID NO: 18 - protein, 113 aa, Artificial Sequence
  Synthetic Polypeptide
DIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGCGTRLEIK

SEQ ID NO: 19 - protein, 113 aa, Artificial Sequence
  Synthetic Polypeptide
DIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGQGTRLEIK

SEQ ID NO: 20 - protein, 505 aa, Artificial Sequence
  Synthetic Polypeptide
QVQLVQSGAEVKKPGESVKVSCKASGYTFTNYGMNWVKQAPGQCLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGCGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 21 - protein, 530 aa, Artificial Sequence
  Synthetic Polypeptide
MGWSCIILFLVATATGVHSQVQLVQSGAEVKKPGESVKVSCKASGYTFTNYGMNWVKQAPGQGLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGQGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 22 - protein, 993 aa, Artificial Sequence
  Synthetic Polypeptide
QVQLVQSGAEVKKPGESVKVSCKASGYTFTNYGMNWVKQAPGQCLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGCGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 511 aa, Artificial Sequence
  Synthetic Polypeptide
QVQLVQSGAEVKKPGESVKVSCKASGYTFTNYGMNWVKQAPGQGLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGQGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 24 - protein, 16 aa, Artificial Sequence
  Synthetic Polypeptide
RSSQSLVHSDGNTYLS

SEQ ID NO: 25 - protein, 7 aa, Artificial Sequence
  Synthetic Polypeptide
RISRRFS

SEQ ID NO: 26 - protein, 9 aa, Artificial Sequence
  Synthetic Polypeptide
MQSTHVPRT

SEQ ID NO: 27 - protein, 5 aa, Artificial Sequence
  Synthetic Polypeptide
NYGMH

SEQ ID NO: 28 - protein, 17 aa, Artificial Sequence
  Synthetic Polypeptide
VIWYDGSDKYYADSVRG

SEQ ID NO: 29 - protein, 15 aa, Artificial Sequence
  Synthetic Polypeptide
DGYDILTGNPRDFDY

SEQ ID NO: 30 - protein, 124 aa, Artificial Sequence
  Synthetic Polypeptide
QVQLVESGGGVVQSGRSLRLSCAASGFTFRNYGMHWVRQAPGKCLEWVAVIWYDGSDKYYADSVRGRFTISRDNSKNTLYLQMNSLRAEDTAVYYCARDGYDILTGNPRDFDYWGQGTLVTVSS

SEQ ID NO: 31 - protein, 112 aa, Artificial Sequence
  Synthetic Polypeptide
DTVMTQTPLSSHVTLGQPASISCRSSQSLVHSDGNTYLSWLQQRPGQPPRLLIYRISRRFSGVPDRFSGSGAGTDFTLEISRVEAEDVGVYYCMQSTHVPRTFGCGTKVEIK

SEQ ID NO: 32 - protein, 251 aa, Artificial Sequence
  Synthetic Polypeptide
QVQLVESGGGVVQSGRSLRLSCAASGFTFRNYGMHWVRQAPGKCLEWVAVIWYDGSDKYYADSVRGRFTISRDNSKNTLYLQMNSLRAEDTAVYYCARDGYDILTGNPRDFDYWGQGTLVTVSSGGGGSGGGGSGGGGSDTVMTQTPLSSHVTLGQPASISCRSSQSLVHSDGNTYLSWLQQRPGQPPRLLIYRISRRFSGVPDRFSGSGAGTDFTLEISRVEAEDVGVYYCMQSTHVPRTFGCGTKVEIK

SEQ ID NO: 33 - protein, 506 aa, Artificial Sequence
  Synthetic Polypeptide
QVQLVESGGGVVQSGRSLRLSCAASGFTFRNYGMHWVRQAPGKCLEWVAVIWYDGSDKYYADSVRGRFTISRDNSKNTLYLQMNSLRAEDTAVYYCARDGYDILTGNPRDFDYWGQGTLVTVSSGGGGSGGGGSGGGGSDTVMTQTPLSSHVTLGQPASISCRSSQSLVHSDGNTYLSWLQQRPGQPPRLLIYRISRRFSGVPDRFSGSGAGTDFTLEISRVEAEDVGVYYCMQSTHVPRTFGCGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 34 - protein, 5 aa, Artificial Sequence
  Synthetic Polypeptide
DYYMT

SEQ ID NO: 35 - protein, 17 aa, Artificial Sequence
  Synthetic Polypeptide
YISSSGSTIYYADSVKG

SEQ ID NO: 36 - protein, 8 aa, Artificial Sequence
  Synthetic Polypeptide
DRNSHFDY

SEQ ID NO: 37 - protein, 11 aa, Artificial Sequence
  Synthetic Polypeptide
RASQGINTWLA

SEQ ID NO: 38 - protein, 7 aa, Artificial Sequence
  Synthetic Polypeptide
GASGLQS

SEQ ID NO: 39 - protein, 9 aa, Artificial Sequence
  Synthetic Polypeptide
QQAKSFPRT

SEQ ID NO: 40 - protein, 117 aa, Artificial Sequence
  Synthetic Polypeptide
QVQLVESGGGLVKPGGSLRLSCAASGFTFSDYYMTWIRQAPGKGLEWLSYISSSGSTIYYADSVKGRFTISRDNAKNSLFLQMNSLRAEDTAVYYCARDRNSHFDYWGQGTLVTVSS

SEQ ID NO: 41 - protein, 107 aa, Artificial Sequence
  Synthetic Polypeptide
DIQMTQSPSSVSASVGDRVTITCRASQGINTWLAWYQQKPGKAPKLLIYGASGLQSGVPSRFSGSGSGTDFTLTISSLQPEDFATYYCQQAKSFPRTFGQGTKVEIK

SEQ ID NO: 42 - protein, 239 aa, Artificial Sequence
  Synthetic Polypeptide
QVQLVESGGGLVKPGGSLRLSCAASGFTFSDYYMTWIRQAPGKGLEWLSYISSSGSTIYYADSVKGRFTISRDNAKNSLFLQMNSLRAEDTAVYYCARDRNSHFDYWGQGTLVTVSSGGGGSGGGGSGGGGSDIQMTQSPSSVSASVGDRVTITCRASQGINTWLAWYQQKPGKAPKLLIYGASGLQSGVPSRFSGSGSGTDFTLTISSLQPEDFATYYCQQAKSFPRTFGQGTKVEIK

SEQ ID NO: 43 - protein, 494 aa, Artificial Sequence
  Synthetic Polypeptide
QVQLVESGGGLVKPGGSLRLSCAASGFTFSDYYMTWIRQAPGKGLEWLSYISSSGSTIYYADSVKGRFTISRDNAKNSLFLQMNSLRAEDTAVYYCARDRNSHFDYWGQGTLVTVSSGGGGSGGGGSGGGGSDIQMTQSPSSVSASVGDRVTITCRASQGINTWLAWYQQKPGKAPKLLIYGASGLQSGVPSRFSGSGSGTDFTLTISSLQPEDFATYYCQQAKSFPRTFGQGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 44 - protein, 982 aa, Artificial Sequence
  Synthetic Polypeptide
QVQLVESGGGLVKPGGSLRLSCAASGFTFSDYYMTWIRQAPGKGLEWLSYISSSGSTIYYADSVKGRFTISRDNAKNSLFLQMNSLRAEDTAVYYCARDRNSHFDYWGQGTLVTVSSGGGGSGGGGSGGGGSDIQMTQSPSSVSASVGDRVTITCRASQGINTWLAWYQQKPGKAPKLLIYGASGLQSGVPSRFSGSGSGTDFTLTISSLQPEDFATYYCQQAKSFPRTFGQGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - protein, 982 aa, Artificial Sequence; identical to SEQ ID NO: 44
  Synthetic Polypeptide

SEQ ID NO: 46 - protein, 5 aa, Artificial Sequence
  Synthetic Polypeptide
SYGMH

SEQ ID NO: 47 - protein, 17 aa, Artificial Sequence
  Synthetic Polypeptide
FIWYEGSNKYYAESVKD

SEQ ID NO: 48 - protein, 16 aa, Artificial Sequence
  Synthetic Polypeptide
RAGIIGTIGYYYGMDV

SEQ ID NO: 49 - protein, 11 aa, Artificial Sequence
  Synthetic Polypeptide
SGDRLGEKYTS

SEQ ID NO: 50 - protein, 7 aa, Artificial Sequence
  Synthetic Polypeptide
QDTKRPS

SEQ ID NO: 51 - protein, 9 aa, Artificial Sequence
  Synthetic Polypeptide
QAWESSTVV

SEQ ID NO: 52 - protein, 125 aa, Artificial Sequence
  Synthetic Polypeptide
QVQLVESGGGVVQPGGSLRLSCAASGFTFSSYGMHWVRQAPGKGLEWVAFIWYEGSNKYYAESVKDRFTISRDNSKNTLYLQMNSLRAEDTAVYYCARRAGIIGTIGYYYGMDVWGQGTTVTVSS

SEQ ID NO: 53 - protein, 107 aa, Artificial Sequence
  Synthetic Polypeptide
SYELTQPPSVSVSPGQTASITCSGDRLGEKYTSWYQQRPGQSPLLVIYQDTKRPSGIPERFSGSNSGNTATLTISGTQAMDEADYYCQAWESSTVVFGGGTKLTVLS

SEQ ID NO: 54 - protein, 247 aa, Artificial Sequence
  Synthetic Polypeptide
QVQLVESGGGVVQPGGSLRLSCAASGFTFSSYGMHWVRQAPGKGLEWVAFIWYEGSNKYYAESVKDRFTISRDNSKNTLYLQMNSLRAEDTAVYYCARRAGIIGTIGYYYGMDVWGQGTTVTVSSGGGGSGGGGSGGGGSSYELTQPPSVSVSPGQTASITCSGDRLGEKYTSWYQQRPGQSPLLVIYQDTKRPSGIPERFSGSNSGNTATLTISGTQAMDEADYYCQAWESSTVVFGGGTKLTVLS

SEQ ID NO: 55 - protein, 507 aa, Artificial Sequence
  Synthetic Polypeptide
QVQLVESGGGVVQPGGSLRLSCAASGFTFSSYGMHWVRQAPGKGLEWVAFIWYEGSNKYYAESVKDRFTISRDNSKNTLYLQMNSLRAEDTAVYYCARRAGIIGTIGYYYGMDVWGQGTTVTVSSGGGGSGGGGSGGGGSSYELTQPPSVSVSPGQTASITCSGDRLGEKYTSWYQQRPGQSPLLVIYQDTKRPSGIPERFSGSNSGNTATLTISGTQAMDEADYYCQAWESSTVVFGGGTKLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 56 - protein, 7 aa, Artificial Sequence
  Synthetic Polypeptide
NARMGVS

SEQ ID NO: 57 - protein, 16 aa, Artificial Sequence
  Synthetic Polypeptide
HIFSNDEKSYSTSLKN

SEQ ID NO: 58 - protein, 14 aa, Artificial Sequence
  Synthetic Polypeptide
IVGYGSGWYGFFDY

SEQ ID NO: 59 - protein, 11 aa, Artificial Sequence
  Synthetic Polypeptide
RASQGIRNDLG

SEQ ID NO: 60 - protein, 7 aa, Artificial Sequence
  Synthetic Polypeptide
AASTLQS

SEQ ID NO: 61 - protein, 9 aa, Artificial Sequence
  Synthetic Polypeptide
LQHNSYPLT

SEQ ID NO: 62 - protein, 124 aa, Artificial Sequence
  Synthetic Polypeptide
QVTLKESGPTLVKPTETLTLTCTLSGFSLNNARMGVSWIRQPPGKCLEWLAHIFSNDEKSYSTSLKNRLTISKDSSKTQVVLTMTNVDPVDTATYYCARIVGYGSGWYGFFDYWGQGTLVTVSS

SEQ ID NO: 63 - protein, 107 aa, Artificial Sequence
  Synthetic Polypeptide
DIQMTQSPSSLSASVGDRVTITCRASQGIRNDLGWYQQKPGKAPKRLIYAASTLQSGVPSRFSGSGSGTEFTLTISSLQPEDFATYYCLQHNSYPLTFGCGTKVEIK

SEQ ID NO: 64 - protein, 246 aa, Artificial Sequence
  Synthetic Polypeptide
QVTLKESGPTLVKPTETLTLTCTLSGFSLNNARMGVSWIRQPPGKCLEWLAHIFSNDEKSYSTSLKNRLTISKDSSKTQVVLTMTNVDPVDTATYYCARIVGYGSGWYGFFDYWGQGTLVTVSSGGGGSGGGGSGGGGSDIQMTQSPSSLSASVGDRVTITCRASQGIRNDLGWYQQKPGKAPKRLIYAASTLQSGVPSRFSGSGSGTEFTLTISSLQPEDFATYYCLQHNSYPLTFGCGTKVEIK

SEQ ID NO: 65 - protein, 501 aa, Artificial Sequence
  Synthetic Polypeptide
QVTLKESGPTLVKPTETLTLTCTLSGFSLNNARMGVSWIRQPPGKCLEWLAHIFSNDEKSYSTSLKNRLTISKDSSKTQVVLTMTNVDPVDTATYYCARIVGYGSGWYGFFDYWGQGTLVTVSSGGGGSGGGGSGGGGSDIQMTQSPSSLSASVGDRVTITCRASQGIRNDLGWYQQKPGKAPKRLIYAASTLQSGVPSRFSGSGSGTEFTLTISSLQPEDFATYYCLQHNSYPLTFGCGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 66 - protein, 989 aa, Artificial Sequence
  Synthetic Polypeptide
QVTLKESGPTLVKPTETLTLTCTLSGFSLNNARMGVSWIRQPPGKCLEWLAHIFSNDEKSYSTSLKNRLTISKDSSKTQVVLTMTNVDPVDTATYYCARIVGYGSGWYGFFDYWGQGTLVTVSSGGGGSGGGGSGGGGSDIQMTQSPSSLSASVGDRVTITCRASQGIRNDLGWYQQKPGKAPKRLIYAASTLQSGVPSRFSGSGSGTEFTLTISSLQPEDFATYYCLQHNSYPLTFGCGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 67 - protein, 5 aa, Artificial Sequence
  Synthetic Polypeptide
SYYWS

SEQ ID NO: 68 - protein, 16 aa, Artificial Sequence
  Synthetic Polypeptide
YVYYSGTTNYNPSLKS

SEQ ID NO: 69 - protein, 10 aa, Artificial Sequence
  Synthetic Polypeptide
IAVTGFYFDY

SEQ ID NO: 70 - protein, 12 aa, Artificial Sequence
  Synthetic Polypeptide
RASQRVNNNYLA

SEQ ID NO: 71 - protein, 7 aa, Artificial Sequence
  Synthetic Polypeptide
GASSRAT

SEQ ID NO: 72 - protein, 9 aa, Artificial Sequence
  Synthetic Polypeptide
QQYDRSPLT

SEQ ID NO: 73 - protein, 118 aa, Artificial Sequence
  Synthetic Polypeptide
QVQLQESGPGLVKPSETLSLTCTVSGGSISSYYWSWIRQPPGKCLEWIGYVYYSGTTNYNPSLKSRVTISVDTSKNQFSLKLSSVTAADTAVYYCASIAVTGFYFDYWGQGTLVTVSS

SEQ ID NO: 74 - protein, 108 aa, Artificial Sequence
  Synthetic Polypeptide
EIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGCGTKLEIK

SEQ ID NO: 75 - protein, 241 aa, Artificial Sequence
  Synthetic Polypeptide
QVQLQESGPGLVKPSETLSLTCTVSGGSISSYYWSWIRQPPGKCLEWIGYVYYSGTTNYNPSLKSRVTISVDTSKNQFSLKLSSVTAADTAVYYCASIAVTGFYFDYWGQGTLVTVSSGGGGSGGGGSGGGGSEIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGCGTKLEIK

SEQ ID NO: 76 - protein, 496 aa, Artificial Sequence
  Synthetic Polypeptide
QVQLQESGPGLVKPSETLSLTCTVSGGSISSYYWSWIRQPPGKCLEWIGYVYYSGTTNYNPSLKSRVTISVDTSKNQFSLKLSSVTAADTAVYYCASIAVTGFYFDYWGQGTLVTVSSGGGGSGGGGSGGGGSEIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 77 - protein, 982 aa, Artificial Sequence
  Synthetic Polypeptide
QVQLQESGPGLVKPSETLSLTCTVSGGSISSYYWSWIRQPPGKCLEWIGYVYYSGTTNYNPSLKSRVTISVDTSKNQFSLKLSSVTAADTAVYYCASIAVTGFYFDYWGQGTLVTVSSGGGGSGGGGSGGGGSEIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 5 aa, Artificial Sequence
  Synthetic Polypeptide
SYGMH

SEQ ID NO: 79 - protein, 17 aa, Artificial Sequence
  Synthetic Polypeptide
VISYEGSNKYYAESVKG

SEQ ID NO: 80 - protein, 13 aa, Artificial Sequence
  Synthetic Polypeptide
DRGTIFGNYGLEV

SEQ ID NO: 81 - protein, 16 aa, Artificial Sequence
  Synthetic Polypeptide
RSSQSLLHKNAFNYLD

SEQ ID NO: 82 - protein, 7 aa, Artificial Sequence
  Synthetic Polypeptide
LGSNRAS

SEQ ID NO: 83 - protein, 9 aa, Artificial Sequence
  Synthetic Polypeptide
MQALQTPFT

SEQ ID NO: 84 - protein, 122 aa, Artificial Sequence
  Synthetic Polypeptide
QVQLVESGGGVVQPGRSLRLSCAASGFTFSSYGMHWVRQAPGKCLEWVAVISYEGSNKYYAESVKGRFTISRDNSKNTLYLQMNSLRDEDTAVYYCARDRGTIFGNYGLEVWGQGTTVTVSS

SEQ ID NO: 85 - protein, 112 aa, Artificial Sequence
  Synthetic Polypeptide
DIVMTQSPLSLPVISGEPASISCRSSQSLLHKNAFNYLDWYLQKPGQSPQLLIYLGSNRASGVPDRFSGSGSGTDFTLKISRVEAEDVGVYYCMQALQTPFTFGCGTKVDIK

SEQ ID NO: 86 - protein, 503 aa, Artificial Sequence
  Synthetic Polypeptide
DIVMTQSPLSLPVISGEPASISCRSSQSLLHKNAFNYLDWYLQKPGQSPQLLIYLGSNRASGVPDRFSGSGSGTDFTLKISRVEAEDVGVYYCMQALQTPFTFGCGTKVDIKGGGGSGGGGSGGGGSQVQLVESGGGVVQPGRSLRLSCAASGFTFSSYGMHWVRQAPGKCLEWVAVISYEGSNKYYAESVKGRFTISRDNSKNTLYLQMNSLRDEDTAVYYCARDRGTIFGNYGLEVWGQGTTVTVSSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 87 - protein, 991 aa, Artificial Sequence
  Synthetic Polypeptide
DIVMTQSPLSLPVISGEPASISCRSSQSLLHKNAFNYLDWYLQKPGQSPQLLIYLGSNRASGVPDRFSGSGSGTDFTLKISRVEAEDVGVYYCMQALQTPFTFGCGTKVDIKGGGGSGGGGSGGGGSQVQLVESGGGVVQPGRSLRLSCAASGFTFSSYGMHWVRQAPGKCLEWVAVISYEGSNKYYAESVKGRFTISRDNSKNTLYLQMNSLRDEDTAVYYCARDRGTIFGNYGLEVWGQGTTVTVSSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 88 - protein, 5 aa, Artificial Sequence
  Synthetic Polypeptide
NHIIH

SEQ ID NO: 89 - protein, 17 aa, Artificial Sequence
  Synthetic Polypeptide
YINPYPGYHAYNEKFQG

SEQ ID NO: 90 - protein, 12 aa, Artificial Sequence
  Synthetic Polypeptide
DGYYRDTDVLDY

SEQ ID NO: 91 - protein, 11 aa, Artificial Sequence
  Synthetic Polypeptide
QASQDISNYLN

SEQ ID NO: 92 - protein, 7 aa, Artificial Sequence
  Synthetic Polypeptide
YTSRLHT

SEQ ID NO: 93 - protein, 9 aa, Artificial Sequence
  Synthetic Polypeptide
QQGNTLPWT

SEQ ID NO: 94 - protein, 121 aa, Artificial Sequence
  Synthetic Polypeptide
QVQLVQSGAEVKKPGASVKVSCKASGYTFTNHIIHWVRQAPGQCLEWMGYINPYPGYHAYNEKFQGRATMTSDTSTSTVYMELSSLRSEDTAVYYCARDGYYRDTDVLDYWGQGTLVTVSS

SEQ ID NO: 95 - protein, 107 aa, Artificial Sequence
  Synthetic Polypeptide
DIQMTQSPSSLSASVGDRVTITCQASQDISNYLNWYQQKPGKAPKLLIYYTSRLHTGVPSRFSGSGSGTDFTFTISSLEPEDIATYYCQQGNTLPWTFGCGTKLEIK

SEQ ID NO: 96 - protein, 243 aa, Artificial Sequence
  Synthetic Polypeptide
QVQLVQSGAEVKKPGASVKVSCKASGYTFTNHIIHWVRQAPGQCLEWMGYINPYPGYHAYNEKFQGRATMTSDTSTSTVYMELSSLRSEDTAVYYCARDGYYRDTDVLDYWGQGTLVTVSSGGGGSGGGGSGGGGSDIQMTQSPSSLSASVGDRVTITCQASQDISNYLNWYQQKPGKAPKLLIYYTSRLHTGVPSRFSGSGSGTDFTFTISSLEPEDIATYYCQQGNTLPWTFGCGTKLEIK

SEQ ID NO: 97 - protein, 986 aa, Artificial Sequence
  Synthetic Polypeptide
QVQLVQSGAEVKKPGASVKVSCKASGYTFTNHIIHWVRQAPGQCLEWMGYINPYPGYHAYNEKFQGRATMTSDTSTSTVYMELSSLRSEDTAVYYCARDGYYRDTDVLDYWGQGTLVTVSSGGGGSGGGGSGGGGSDIQMTQSPSSLSASVGDRVTITCQASQDISNYLNWYQQKPGKAPKLLIYYTSRLHTGVPSRFSGSGSGTDFTFTISSLEPEDIATYYCQQGNTLPWTFGCGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 98 - protein, 504 aa, Artificial Sequence
  Synthetic Polypeptide
QVQLVQSGAEVKKPGASVKVSCKASGYTFTNHIIHWVRQAPGQCLEWMGYINPYPGYHAYNEKFQGRATMTSDTSTSTVYMELSSLRSEDTAVYYCARDGYYRDTDVLDYWGQGTLVTVSSGGGGSGGGGSGGGGSDIQMTQSPSSLSASVGDRVTITCQASQDISNYLNWYQQKPGKAPKLLIYYTSRLHTGVPSRFSGSGSGTDFTFTISSLEPEDIATYYCQQGNTLPWTFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 99 - protein, 986 aa, Artificial Sequence; identical to SEQ ID NO: 97
  Synthetic Polypeptide

SEQ ID NO: 100 - protein, 5 aa, Artificial Sequence
  Synthetic Polypeptide
DYYMY

SEQ ID NO: 101 - protein, 17 aa, Artificial Sequence
  Synthetic Polypeptide
IISDAGYYTYYSDIIKG

SEQ ID NO: 102 - protein, 12 aa, Artificial Sequence
  Synthetic Polypeptide
GFPLLRHGAMDY

SEQ ID NO: 103 - protein, 11 aa, Artificial Sequence
  Synthetic Polypeptide
KASQNVDANVA

SEQ ID NO: 104 - protein, 7 aa, Artificial Sequence
  Synthetic Polypeptide
SASYVYW

SEQ ID NO: 105 - protein, 9 aa, Artificial Sequence
  Synthetic Polypeptide
QQYDQQLIT

SEQ ID NO: 106 - protein, 121 aa, Artificial Sequence
  Synthetic Polypeptide
QVQLVESGGGLVKPGESLRLSCAASGFTFSDYYMYWVRQAPGKCLEWVAIISDAGYYTYYSDIIKGRFTISRDNAKNSLYLQMNSLKAEDTAVYYCARGFPLLRHGAMDYWGQGTLVTVSS

SEQ ID NO: 107 - protein, 107 aa, Artificial Sequence
  Synthetic Polypeptide
DIQMTQSPSSLSASVGDRVTITCKASQNVDANVAWYQQKPGQAPKSLIYSASYVYWDVPSRFSGSASGTDFTLTISSVQSEDFATYYCQQYDQQLITFGCGTKLEIK

SEQ ID NO: 108 - protein, 243 aa, Artificial Sequence
  Synthetic Polypeptide
QVQLVESGGGLVKPGESLRLSCAASGFTFSDYYMYWVRQAPGKCLEWVAIISDAGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDANVAWYQQKPGQAPKSLIYSASYVYWDVPSRFSGSASGTDFTLTISSVQSEDFATYYCQQYDQQLITFGCGTKLEIK

SEQ ID NO: 109 - protein, 498 aa, Artificial Sequence
  Synthetic Polypeptide
QVQLVESGGGLVKPGESLRLSCAASGFTFSDYYMYWVRQAPGKGLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYRYSDVPSRFSGSASGTDFTLTISSVQSEDFATYYCQQYDSYPYTFGGGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 110 - protein, 986 aa, Artificial Sequence
  Synthetic Polypeptide
QVQLVESGGGLVKPGESLRLSCAASGFTFSDYYMYWVRQAPGKCLEWVAIISDAGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDANVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 111 - protein, 984 aa, Artificial Sequence
  Synthetic Polypeptide
QVQLVESGGGLVKPGESLRLSCAASGFTFSDYYMYWVRQAPGKCLEWVAIISDAGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDANVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 112 - protein, 5 aa, Artificial Sequence
  Synthetic Polypeptide
DYYMY

SEQ ID NO: 113 - protein, 17 aa, Artificial Sequence
  Synthetic Polypeptide
IISDGGYYTYYSDIIKG

SEQ ID NO: 114 - protein, 12 aa, Artificial Sequence
  Synthetic Polypeptide
GFPLLRHGAMDY

SEQ ID NO: 115 - protein, 11 aa, Artificial Sequence
  Synthetic Polypeptide
KASQNVDTNVA

SEQ ID NO: 116 - protein, 7 aa, Artificial Sequence
  Synthetic Polypeptide
SASYVYW

SEQ ID NO: 117 - protein, 9 aa, Artificial Sequence
  Synthetic Polypeptide
QQYDQQLIT

SEQ ID NO: 118 - protein, 121 aa, Artificial Sequence
  Synthetic Polypeptide
QVQLVESGGGLVKPGESLRLSCAASGFTFSDYYMYWVRQAPGKGLEWVAIISDGGYYTYYSDIIKGRFTISRDNAKNSLYLQMNSLKAEDTAVYYCARGFPLLRHGAMDYWGQGTLVTVSS

SEQ ID NO: 119 - protein, 107 aa, Artificial Sequence
  Synthetic Polypeptide
DIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGGGTKLEIK

SEQ ID NO: 120 - protein, 243 aa, Artificial Sequence
  Synthetic Polypeptide
QVQLVESGGGLVKPGESLRLSCAASGFTFSDYYMYWVRQAPGKGLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGGGTKLEIK

SEQ ID NO: 121 - protein, 498 aa, Artificial Sequence
  Synthetic Polypeptide
QVQLVESGGGLVKPGESLRLSCAASGFTFSDYYMYWVRQAPGKGLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGGGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 122 - protein, 986 aa, Artificial Sequence
  Synthetic Polypeptide
QVQLVESGGGLVKPGESLRLSCAASGFTFSDYYMYWVRQAPGKCLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 123 - protein, 5 aa, Artificial Sequence
  Synthetic Polypeptide
TYAMS

SEQ ID NO: 124 - protein, 17 aa, Artificial Sequence
  Synthetic Polypeptide
AISGSGGRTFYAESVEG

SEQ ID NO: 125 - protein, 11 aa, Artificial Sequence
  Synthetic Polypeptide
HDYSNYPYFDY

SEQ ID NO: 126 - protein, 12 aa, Artificial Sequence
  Synthetic Polypeptide
RASQSVRSTYLA

SEQ ID NO: 127 - protein, 7 aa, Artificial Sequence
  Synthetic Polypeptide
GASSRAT

SEQ ID NO: 128 - protein, 9 aa, Artificial Sequence
  Synthetic Polypeptide
QQYGDLPFT

SEQ ID NO: 129 - protein, 120 aa, Artificial Sequence
  Synthetic Polypeptide
EVQLLESGGGMVQPGGSLRLSCAASGFTFSTYAMSWVRQAPGKCLEWVSAISGSGGRTFYAESVEGRFTISRDNSKNTLYLQMNSLRAEDTAVYYCAKHDYSNYPYFDYWGQGTLVTVSS

SEQ ID NO: 130 - protein, 108 aa, Artificial Sequence
  Synthetic Polypeptide
EIVLTQSPGTLSLSPGERATLSCRASQSVRSTYLAWYQQKPGQAPRLLIYGASSRATGIPDRFSGSGSGTDFTLTISRLEPEDFAVYSCQQYGDLPFTFGCGTKLEIK

SEQ ID NO: 131 - protein, 986 aa, Artificial Sequence
  Synthetic Polypeptide
EVQLLESGGGMVQPGGSLRLSCAASGFTFSTYAMSWVRQAPGKCLEWVSAISGSGGRTFYAESVEGRFTISRDNSKNTLYLQMNSLRAEDTAVYYCAKHDYSNYPYFDYWGQGTLVTVSSGGGGSGGGGSGGGGSEIVLTQSPGTLSLSPGERATLSCRASQSVRSTYLAWYQQKPGQAPRLLIYGASSRATGIPDRFSGSGSGTDFTLTISRLEPEDFAVYSCQQYGDLPFTFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 132 - protein, 5 aa, Artificial Sequence
  Synthetic Polypeptide
GYYMH

SEQ ID NO: 133 - protein, 17 aa, Artificial Sequence
  Synthetic Polypeptide
WINPNSGGTKYAQKFQG

SEQ ID NO: 134 - protein, 16 aa, Artificial Sequence
  Synthetic Polypeptide
DRITVAGTYYYYGMDV

SEQ ID NO: 135 - protein, 11 aa, Artificial Sequence
  Synthetic Polypeptide
RASQGVNNWLA

SEQ ID NO: 136 - protein, 7 aa, Artificial Sequence
  Synthetic Polypeptide
TASSLQS

SEQ ID NO: 137 - protein, 9 aa, Artificial Sequence
  Synthetic Polypeptide
QQANSFPIT

SEQ ID NO: 138 - protein, 125 aa, Artificial Sequence
  Synthetic Polypeptide
QVQLVQSGAEVKKPGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSS

SEQ ID NO: 139 - protein, 107 aa, Artificial Sequence
  Synthetic Polypeptide
DIQMTQSPSSVSASVGDRVTITCRASQGVNNWLAWYQQKPGKAPKLLIYTASSLQSGVPSRFSGSGSGTDFTLTIRSLQPEDFATYYCQQANSFPITFGCGTRLEIK

SEQ ID NO: 140 - protein, 247 aa, Artificial Sequence
  Synthetic Polypeptide
QVQLVQSGAEVKKPGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSSGGGGSGGGGSGGGGSDIQMTQSPSSVSASVGDRVTITCRASQGVNNWLAWYQQKPGKAPKLLIYTASSLQSGVPSRFSGSGSGTDFTLTIRSLQPEDFATYYCQQANSFPITFGCGTRLEIK

SEQ ID NO: 141 - protein, 502 aa, Artificial Sequence
  Synthetic Polypeptide
QVQLVQSGAEVKKPGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSSGGGGSGGGGSGGGGSDIQMTQSPSSVSASVGDRVTITCRASQGVNNWLAWYQQKPGKAPKLLIYTASSLQSGVPSRFSGSGSGTDFTLTIRSLQPEDFATYYCQQANSFPITFGCGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 142 - protein, 990 aa, Artificial Sequence
  Synthetic Polypeptide
QVQLVQSGAEVKKPGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSSGGGGSGGGGSGGGGSDIQMTQSPSSVSASVGDRVTITCRASQGVNNWLAWYQQKPGKAPKLLIYTASSLQSGVPSRFSGSGSGTDFTLTIRSLQPEDFATYYCQQANSFPITFGCGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 143 - protein, 125 aa, Artificial Sequence
  Synthetic Polypeptide
QVQMVQSGAEVKKHGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSS

SEQ ID NO: 144 - protein, 107 aa, Artificial Sequence; identical to SEQ ID NO: 139
  Synthetic Polypeptide

SEQ ID NO: 145 - protein, 247 aa, Artificial Sequence
  Synthetic Polypeptide
QVQMVQSGAEVKKHGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSSGGGGSGGGGSGGGGSDIQMTQSPSSVSASVGDRVTITCRASQGVNNWLAWYQQKPGKAPKLLIYTASSLQSGVPSRFSGSGSGTDFTLTIRSLQPEDFATYYCQQANSFPITFGCGTRLEIK

SEQ ID NO: 146 - protein, 502 aa, Artificial Sequence
  Synthetic Polypeptide
QVQMVQSGAEVKKHGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSSGGGGSGGGGSGGGGSDIQMTQSPSSVSASVGDRVTITCRASQGVNNWLAWYQQKPGKAPKLLIYTASSLQSGVPSRFSGSGSGTDFTLTIRSLQPEDFATYYCQQANSFPITFGCGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 147 - protein, 990 aa, Artificial Sequence
  Synthetic Polypeptide
QVQMVQSGAEVKKHGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSSGGGGSGGGGSGGGGSDIQMTQSPSSVSASVGDRVTITCRASQGVNNWLAWYQQKPGKAPKLLIYTASSLQSGVPSRFSGSGSGTDFTLTIRSLQPEDFATYYCQQANSFPITFGCGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 148 - protein, 5 aa, Artificial Sequence
  Synthetic Polypeptide
NHGMH

SEQ ID NO: 149 - protein, 17 aa, Artificial Sequence
  Synthetic Polypeptide
GIWSEGSNKYYADAVKG

SEQ ID NO: 150 - protein, 12 aa, Artificial Sequence
  Synthetic Polypeptide
ATYTTGWSYFDY

SEQ ID NO: 151 - protein, 11 aa, Artificial Sequence
  Synthetic Polypeptide
SGDKLGDKYAS

SEQ ID NO: 152 - protein, 7 aa, Artificial Sequence
  Synthetic Polypeptide
QDAKRPS

SEQ ID NO: 153 - protein, 9 aa, Artificial Sequence
  Synthetic Polypeptide
QAFHQSTWV

SEQ ID NO: 154 - protein, 121 aa, Artificial Sequence
  Synthetic Polypeptide
QVQLVESGGGVVQPGRSLRLSCAASGFTFSNHGMHWVRQAPGKCLEWVAGIWSEGSNKYYADAVKGRFTISRDNSKNTLYLQMNSLRAEDTAVYYCARATYTTGWSYFDYWGQGTLVTVSS

SEQ ID NO: 155 - protein, 106 aa, Artificial Sequence
  Synthetic Polypeptide
SYELTQPPSVSVSPGQTASITCSGDKLGDKYASWYQQKSGQSPVLVIYQDAKRPSGIPERFSGSNSGNTATLTISGTQAMDEADYYCQAFHQSTWVFGCGTQLTVL

SEQ ID NO: 156 - protein, 985 aa, Artificial Sequence
  Synthetic Polypeptide
QVQLVESGGGVVQPGRSLRLSCAASGFTFSNHGMHWVRQAPGKCLEWVAGIWSEGSNKYYADAVKGRFTISRDNSKNTLYLQMNSLRAEDTAVYYCARATYTTGWSYFDYWGQGTLVTVSSGGGGSGGGGSGGGGSSYELTQPPSVSVSPGQTASITCSGDKLGDKYASWYQQKSGQSPVLVIYQDAKRPSGIPERFSGSNSGNTATLTISGTQAMDEADYYCQAFHQSTWVFGCGTQLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 157 - protein, 5 aa, Artificial Sequence
  Synthetic Polypeptide
NHAMH

SEQ ID NO: 158 - protein, 17 aa, Artificial Sequence
  Synthetic Polypeptide
GIWSEGSNKYYAESVKG

SEQ ID NO: 159 - protein, 12 aa, Artificial Sequence
  Synthetic Polypeptide
ATYTTGWSYFDY

SEQ ID NO: 160 - protein, 11 aa, Artificial Sequence
  Synthetic Polypeptide
SGDKLGDKYAS

SEQ ID NO: 161 - protein, 7 aa, Artificial Sequence
  Synthetic Polypeptide
QDRKRPS

SEQ ID NO: 162 - protein, 9 aa, Artificial Sequence
  Synthetic Polypeptide
QAYDASTWV

SEQ ID NO: 163 - protein, 121 aa, Artificial Sequence
  Synthetic Polypeptide
QVQLVESGGGVVQPGRSLRLSCAASGFTFSNHAMHWVRQAPGKCLEWVAGIWSEGSNKYYAESVKGRFTISRDNSKNTLYLQMNSLRAEDTAVYYCARATYTTGWSYFDYWGQGTLVTVSS

SEQ ID NO: 164 - protein, 106 aa, Artificial Sequence
  Synthetic Polypeptide
SYELTQPPSVSVSPGQTASITCSGDKLGDKYASWYQQKSGQSPVLVIYQDRKRPSGIPERFSGSNSGNTATLTISGTQAMDEADYYCQAYDASTWVFGCGTQLTVL

SEQ ID NO: 165 - protein, 985 aa, Artificial Sequence
  Synthetic Polypeptide
QVQLVESGGGVVQPGRSLRLSCAASGFTFSNHAMHWVRQAPGKCLEWVAGIWSEGSNKYYAESVKGRFTISRDNSKNTLYLQMNSLRAEDTAVYYCARATYTTGWSYFDYWGQGTLVTVSSGGGGSGGGGSGGGGSSYELTQPPSVSVSPGQTASITCSGDKLGDKYASWYQQKSGQSPVLVIYQDRKRPSGIPERFSGSNSGNTATLTISGTQAMDEADYYCQAYDASTWVFGCGTQLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 166 - protein, 5 aa, Artificial Sequence
  Synthetic Polypeptide
GYYWS

SEQ ID NO: 167 - protein, 16 aa, Artificial Sequence
  Synthetic Polypeptide
DIDASGSTKYNPSLKS

SEQ ID NO: 168 - protein, 12 aa, Artificial Sequence
  Synthetic Polypeptide
KKYSTVWSYFDN

SEQ ID NO: 169 - protein, 11 aa, Artificial Sequence
  Synthetic Polypeptide
SGDKLGDKYAS

SEQ ID NO: 170 - protein, 7 aa, Artificial Sequence
  Synthetic Polypeptide
QDRKRPS

SEQ ID NO: 171 - protein, 9 aa, Artificial Sequence
  Synthetic Polypeptide
QAWGSSTAV

SEQ ID NO: 172 - protein, 120 aa, Artificial Sequence
  Synthetic Polypeptide
QVQLQQWGAGLLKPSETLSLTCAVYGGSFSGYYWSWIRQPPGKCLEWIGDIDASGSTKYNPSLKSRVTISLDTSKNQFSLKLNSVTAADTAVYFCARKKYSTVWSYFDNWGQGTLVTVSS

SEQ ID NO: 173 - protein, 106 aa, Artificial Sequence
  Synthetic Polypeptide
SYELTQPSSVSVPPGQTASITCSGDKLGDKYASWYQQKPGQSPVLVIYQDRKRPSGVPERFSGSNSGNTATLTISGTQAMDEADYYCQAWGSSTAVFGCGTKLTVL

SEQ ID NO: 174 - protein, 984 aa, Artificial Sequence
  Synthetic Polypeptide
QVQLQQWGAGLLKPSETLSLTCAVYGGSFSGYYWSWIRQPPGKCLEWIGDIDASGSTKYNPSLKSRVTISLDTSKNQFSLKLNSVTAADTAVYFCARKKYSTVWSYFDNWGQGTLVTVSSGGGGSGGGGSGGGGSSYELTQPSSVSVPPGQTASITCSGDKLGDKYASWYQQKPGQSPVLVIYQDRKRPSGVPERFSGSNSGNTATLTISGTQAMDEADYYCQAWGSSTAVFGCGTKLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 175 - protein, 5 aa, Artificial Sequence
  Synthetic Polypeptide
GYYWS

SEQ ID NO: 176 - protein, 16 aa, Artificial Sequence
  Synthetic Polypeptide
DIDYSGSTKYNPSLKS

SEQ ID NO: 177 - protein, 12 aa, Artificial Sequence
  Synthetic Polypeptide
KKYSTVWSYFDY

SEQ ID NO: 178 - protein, 11 aa, Artificial Sequence
  Synthetic Polypeptide
SGDKLGDKYAN

SEQ ID NO: 179 - protein, 7 aa, Artificial Sequence
  Synthetic Polypeptide
HDNKRPS

SEQ ID NO: 180 - protein, 9 aa, Artificial Sequence
  Synthetic Polypeptide
QAYGISSAV

SEQ ID NO: 181 - protein, 120 aa, Artificial Sequence
  Synthetic Polypeptide
QVQLQQWGAGLLKPSETLSLTCAVYGGSFSGYYWSWIRQPPGKCLEWIGDIDYSGSTKYNPSLKSRVTISLDTSKNQFSLKLNSVTAADTAVYFCARKKYSTVWSYFDYWGQGTLVTVSS

SEQ ID NO: 182 - protein, 106 aa, Artificial Sequence
  Synthetic Polypeptide
SYELTQPASASVSPGQTASITCSGDKLGDKYANWYQQKPGQSPILVIYHDNKRPSGIPERFSGSNSGNTATLTISGTQAMDEADYYCQAYGISSAVFGCGTKLTVL

SEQ ID NO: 183 - protein, 984 aa, Artificial Sequence
  Synthetic Polypeptide
QVQLQQWGAGLLKPSETLSLTCAVYGGSFSGYYWSWIRQPPGKCLEWIGDIDYSGSTKYNPSLKSRVTISLDTSKNQFSLKLNSVTAADTAVYFCARKKYSTVWSYFDYWGQGTLVTVSSGGGGSGGGGSGGGGSSYELTQPASASVSPGQTASITCSGDKLGDKYANWYQQKPGQSPILVIYHDNKRPSGIPERFSGSNSGNTATLTISGTQAMDEADYYCQAYGISSAVFGCGTKLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 184 - protein, 989 aa, Artificial Sequence
  Synthetic Polypeptide
QVQLVESGGGVVQPGGSLRLSCAASGFTFSSYGMHWVRQAPGKGLEWVAFIWYEGSNKYYAESVKDRFTISRDNSKNTLYLQMNSLRAEDTAVYYCARRAGIIGTIGYYYGMDVWGQGTTVTVSSGGGGSGGGGSGGGGSSYELTQPPSVSVSPGQTASITCSGDRLGEKYTSWYQQRPGQSPLLVIYQDTKRPSGIPERFSGSNSGNTATLTISGTQAMDEADYYCQAWESSTVVFGGGTKLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 185 - protein, 987 aa, Artificial Sequence
  Synthetic Polypeptide
QVQLVESGGGVVQPGGSLRLSCAASGFTFSSYGMHWVRQAPGKGLEWVAFIWYEGSNKYYAESVKDRFTISRDNSKNTLYLQMNSLRAEDTAVYYCARRAGIIGTIGYYYGMDVWGQGTTVTVSSGGGGSGGGGSGGGGSSYELTQPPSVSVSPGQTASITCSGDRLGEKYTSWYQQRPGQSPLLVIYQDTKRPSGIPERFSGSNSGNTATLTISGTQAMDEADYYCQAWESSTVVFGGGTKLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 186 - protein, 501 aa, Artificial Sequence
  Synthetic Polypeptide
QVQLVESGGGVVQPGGSLRLSCAASGFTFSSYGMHWVRQAPGKCLEWVAFIWYEGSNKYYAESVKDRFTISRDNSKNTLYLQMNSLRAEDTAVYYCARRAGIIGTIGYYYGMDVWGQGTTVTVSSGGGGSGGGGSGGGGSSYELTQPPSVSVSPGQTASITCSGDRLGEKYTSWYQQRPGQSPLLVIYQDTKRPSGIPERFSGSNSGNTATLTISGTQAMDEADYYCQAWESSTVVFGCGTKLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 187 - protein, 989 aa, Artificial Sequence
  Synthetic Polypeptide
QVQLVESGGGVVQPGGSLRLSCAASGFTFSSYGMHWVRQAPGKCLEWVAFIWYEGSNKYYAESVKDRFTISRDNSKNTLYLQMNSLRAEDTAVYYCARRAGIIGTIGYYYGMDVWGQGTTVTVSSGGGGSGGGGSGGGGSSYELTQPPSVSVSPGQTASITCSGDRLGEKYTSWYQQRPGQSPLLVIYQDTKRPSGIPERFSGSNSGNTATLTISGTQAMDEADYYCQAWESSTVVFGCGTKLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 188 - protein, 987 aa, Artificial Sequence
  Synthetic Polypeptide
QVQLVESGGGVVQPGGSLRLSCAASGFTFSSYGMHWVRQAPGKCLEWVAFIWYEGSNKYYAESVKDRFTISRDNSKNTLYLQMNSLRAEDTAVYYCARRAGIIGTIGYYYGMDVWGQGTTVTVSSGGGGSGGGGSGGGGSSYELTQPPSVSVSPGQTASITCSGDRLGEKYTSWYQQRPGQSPLLVIYQDTKRPSGIPERFSGSNSGNTATLTISGTQAMDEADYYCQAWESSTVVFGCGTKLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 189 - protein, 250 aa, Artificial Sequence
  Synthetic Polypeptide
QVQLVQSGAEVKKPGESVKVSCKASGYTFTNYGMNWVKQAPGQCLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGCGTRLEIK

SEQ ID NO: 190 - protein, 250 aa, Artificial Sequence
  Synthetic Polypeptide
QVQLVQSGAEVKKPGESVKVSCKASGYTFTNYGMNWVKQAPGQGLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGQGTRLEIK

SEQ ID NO: 191 - protein, 244 aa, Artificial Sequence
  Synthetic Polypeptide
EVQLLESGGGMVQPGGSLRLSCAASGFTFSTYAMSWVRQAPGKCLEWVSAISGSGGRTFYAESVEGRFTISRDNSKNTLYLQMNSLRAEDTAVYYCAKHDYSNYPYFDYWGQGTLVTVSSGGGGSGGGGSGGGGSEIVLTQSPGTLSLSPGERATLSCRASQSVRSTYLAWYQQKPGQAPRLLIYGASSRATGIPDRFSGSGSGTDFTLTISRLEPEDFAVYSCQQYGDLPFTFGCGTKLEIKS

SEQ ID NO: 192 - protein, 250 aa, Artificial Sequence
  Synthetic Polypeptide
SEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 193 - protein, 484 aa, Artificial Sequence
  Synthetic Polypeptide
DKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 194 - protein, 242 aa, Artificial Sequence
  Synthetic Polypeptide
QVQLQQWGAGLLKPSETLSLTCAVYGGSFSGYYWSWIRQPPGKCLEWIGDIDASGSTKYNPSLKSRVTISLDTSKNQFSLKLNSVTAADTAVYFCARKKYSTVWSYFDNWGQGTLVTVSSGGGGSGGGGSGGGGSSYELTQPSSVSVPPGQTASITCSGDKLGDKYASWYQQKPGQSPVLVIYQDRKRPSGVPERFSGSNSGNTATLTISGTQAMDEADYYCQAWGSSTAVFGCGTKLTVLS

SEQ ID NO: 195 - protein, 250 aa, Artificial Seuqence; identical to SEQ ID NO: 192
  Synthetic Polypeptide

SEQ ID NO: 196 - protein, 484 aa, Artificial Sequence; identical to SEQ ID NO: 193
  Synthetic Polypeptide

SEQ ID NO: 197 - protein, 4 aa, Artificial Sequence
  Synthetic Linker
GGGG

(19) United States
(12) Patent Application Publication
(10) Pub. No.: US 2024/0316194 A1
(43) Pub. Date: Sep. 26, 2024

(54) METHOD OF RECONSTITUTING LYOPHILIZED FORMULATION

(71) Applicant: AMGEN INC., Thousand Oaks, CA (US)

(72) Inventors: Wei Qi, Thousand Oaks, CA (US); Cindy Ren, Calabasas, CA (US)

(21) Appl. No.: 18/574,161

(22) PCT Filed: Jun. 29, 2022
(86) PCT No.: PCT/US22/35533

§ 371 Date: Dec. 26, 2023

Related U.S. Application Data
(60) Provisional application No. 63/216,616, filed on Jun. 30, 2021.

(51) Int. Cl. A61K 39/395; A61K 9/00; A61K 9/19; A61K 39/00; A61K 47/10; A61K 47/18; A61K 47/26; C07K 16/28; C07K 16/30

(52) U.S. Cl. CPC A61K 39/39591; A61K 9/0019; A61K 9/19; A61K 47/10; A61K 47/183; A61K 47/26; C07K 16/2809; C07K 16/3092; A61K 2039/505; C07K 2317/31; C07K 2317/94

(57) ABSTRACT

Described herein is a method comprising (a) reconstituting a lyophilized formulation comprising a polypeptide, a saccharide, a surfactant, and a buffer, with a diluent comprising an aromatic alcohol, a phenolic compound, or an amino acid with aromatic side chain, and (b) administering the reconstituted formulation to a subject in need thereof within 12 hours of reconstitution, wherein the polypeptide is a bispecific antibody construct, such as a bispecific antibody construct that binds human DLL3 and human CD3 and optionally comprises the amino acid sequence of SEQ ID NO: 75 and SEQ ID NO: 9.

### CROSS REFERENCE TO RELATED APPLICATIONS AND INCORPORATION BY REFERENCE

This application claims the benefit of U.S. Provisional Application No. 63/216,616, filed Jun. 30, 2021, the entire contents of which are fully incorporated herein by reference

Incorporated by reference in its entirety is a computer-readable nucleotide/amino acid sequence listing submitted concurrently herewith and identified as follows: ASCII (text) file named “56395_Seqlisting.txt”, 362,780 bytes, created Jun. 29, 2022.

### FIELD OF THE INVENTION

The present disclosure is in the field of protein therapeutics and methods of use.

### BACKGROUND

Protein-based pharmaceuticals, such as recombinant proteins, can now be obtained in high purity when first manufactured due to advances in commercial scale purification processes. However, proteins are only marginally stable and are highly susceptible to degradation, both chemical and physical. Chemical degradation refers to modifications involving covalent bonds, such as deamidation, oxidation, cleavage, clipping/fragmentation, formation of new disulfide bridges, hydrolysis, isomerization, or deglycosylation. Physical degradation includes protein unfolding, undesirable adsorption to surfaces, and aggregation. Dealing with these physical and chemical instabilities is one of the most challenging tasks in the development of protein pharmaceuticals (Chi et al., Pharm Res, Vol. 20, No. 9, Sept 2003, pp. 1325-1336; Roberts, Trends Biotechnol. 2014 July;32(7):372-80).

Aggregation is a significant type of physical instability observed in protein formulations. It is the assembly from initially native and folded proteins into high molecular weight species (HMW). Half-life extended bispecific T cell engaging, antigen binding polypeptides, such as, e.g., bispecific T cell engager (BiTE®) molecules comprising a half-life extending modality such as Fc-molecules, are prone to protein aggregation and/or other degradation events.

Protein aggregation of BiTE® molecules is problematic because it can impair biological activity of the therapeutic proteins. Further, there is concern that the presence of aggregated proteins (even humanized or fully human proteins) can increase the risk that a patient will develop an immune response to the active protein monomer, resulting in the formation of neutralizing antibodies and drug resistance or other adverse side effects (Mahler J Pharm Sci. 2009 September;98(9):2909-34). While protein aggregates are removed as impurities in, e.g., the polishing steps of downstream processing, aggregates can form after shipment to clinical sites and handling by clinicians.

There is a need in the art for formulations and methods to minimize the amount of high molecular weight species in formulations prepared for administration to patients and associated inconvenience in working with protein therapeutics at the point of care.

### SUMMARY

The present disclosure provides a method comprising (a) reconstituting a lyophilized formulation comprising a polypeptide, a saccharide, a surfactant, and a buffer, with a diluent comprising an aromatic alcohol, a phenolic compound, or an amino acid with aromatic side chain, and (b) administering the reconstituted formulation to a subject in need thereof within 12 hours of reconstitution. In various aspects, the polypeptide is a bispecific antibody construct. In various aspects, the polypeptide binds human DLL3 and human CD3, such as a polypeptide comprising the amino acid sequence of SEQ ID NO: 75 and SEQ ID NO: 9. Optionally, the diluent comprises benzyl alcohol, phenol, m-Cresol, or phenylalanine. Also optionally, the pH of the reconstituted formulation is from about 4 to about 7 and/or the reconstituted formulation comprises about 1 mg/mL to about 20 mg/mL of the polypeptide. In various aspects, the buffer is a glutamate buffer, the saccharide is sucrose, and/or the surfactant is polysorbate 80. For example, in an exemplary aspect, the formulation, prior to lyophilization, comprises 15 mM glutamate, 9% (wN) sucrose and 0.01% (w/V) polysorbate 80, wherein the formulation has a pH of 4.2. In various aspects, the subject is suffering from cancer, such as small cell lung cancer (SCLC), non-small cell lung cancer (NSCLC), glioma, glioblastoma, melanoma, neuroendocrine prostate cancer, neuroendocrine pancreatic cancer, hepatoblastoma, or hepatocellular carcinoma.

It should be understood that while various embodiments in the specification are presented using “comprising” language, under various circumstances, a related embodiment may also be described using “consisting of or “consisting essentially of” language. The disclosure contemplates embodiments described as “comprising” a feature to include embodiments which “consist of” or “consists essentially of” the feature. The term “a” or “an” refers to one or more; for example, “an immunoglobulin molecule,” is understood to represent one or more immunoglobulin molecules. As such, the terms “a” (or “an”), “one or more,” and “at least one” can be used interchangeably herein.

It should also be understood that when describing a range of values, the characteristic being described could be an individual value found within the range. For example, “a pH from about pH 4 to about pH 6,” could be, but is not limited to, pH 4, 4.2, 4.6, 5.1, 5.5 etc. and any value in between such values. Additionally, “a pH from about pH 4 to about pH 6,” should not be construed to mean that the pH of a formulation in question varies 2 pH units in the range from pH 4 to pH 6 during storage, but rather a value may be picked in that range for the pH of the solution, and the pH remains buffered at about that pH. In any of the ranges described herein, the endpoints of the range are included in the range. However, the description also contemplates the same ranges in which the lower and/or the higher endpoint is excluded.

When the term “about” is used, it means the recited number plus or minus 5%, 10%, 15% or more of that recited number. The actual variation intended is determinable from the context.

Additional features and variations of the invention will be apparent to those skilled in the art from the entirety of this application, including the drawings, detailed description, and appended claims, and all such features are intended as aspects of the disclosure. Likewise, features of the invention described herein can be re-combined into additional embodiments that also are intended as aspects of the invention, irrespective of whether the combination of features is specifically mentioned above as an aspect or embodiment of the invention. Also, only such limitations which are described herein as critical to the invention should be viewed as such; variations of the invention lacking limitations which have not been described herein as critical are intended as aspects of the invention.

All of the references cited herein, including patents, patent applications, literature publications, and the like, are hereby incorporated in their entireties by reference.

### BRIEF DESCRIPTION OF THE FIGURES

FIG. 1 is a line graph illustrating percentage of high molecular weight species (HMW %) identified by size exclusion chromatograph (SEC) (y-axis) over time (hours) (x-axis) following reconstitution of a lyophilized formulation using sterile water for injection (sWFI, dark circles), WFI comprising 0.9% benzyl alcohol (bWFI, dark triangles), saline (“x”), saline comprising 0.9% benzyl alcohol (open triangles), polysorbate 80 (open diamonds), or intravenous solution stabilizer (IVSS, dark diamonds). The study was performed at 8° C.

FIG. 2 is a line graph illustrating percentage of high molecular weight species (HMW %) identified by size exclusion chromatograph (SEC) (y-axis) over time (hours) (x-axis) following reconstitution of a lyophilized formulation at 8° C. and 25° C. using sterile water for injection (sWFI, dark circles=8° C., open circles=25° C.), phenylalanine (dark squares=8° C., open squares=25° C.), or WFI comprising 0.9% benzyl alcohol (bWFI, dark triangles=8° C., open triangles=25° C.).

FIG. 3 is a line graph illustrating percentage of high molecular weight species (HMW %) identified by size exclusion chromatograph (SEC) (y-axis) over time (hours) (x-axis) following reconstitution of a lyophilized formulation using sterile water for injection (sWFI, dark circles), phenylalanine (Phe, dark squares), phenol (“x”), m-cresol (dark diamonds), or WFI comprising 0.9% benzyl alcohol (bWFI, dark triangles). The study was performed at 8° C.

FIG. 4 is a line graph illustrating percentage of high molecular weight species (HMW %) identified by size exclusion chromatograph (SEC) (y-axis) over time (minutes) (x-axis) following reconstitution of a lyophilized formulation using WFI comprising 0.9% benzyl alcohol. The study was performed at 25° C. The SEC measurement was performed at 8° C. The two lines represent separate analyses performed on two lyophilized samples.

FIG. 5 is a bar graph illustrating the effects of different concentrations of benzyl alcohol on the dissociation of high molecular weight species following reconstitution of a lyophilized BiTE® molecule comprising SEQ ID NOs: 75 and 9. Benzyl alcohol concentration is noted on the x-axis (1.0 mM, 10.0 mM, 41.6 mM, and 83.2 mM), while t_{90%} hr is noted on the y-axis.

FIG. 6 is a table listing various sequences referenced in the disclosure.

### DETAILED DESCRIPTION

Despite the high quality of current therapeutic biotech products and the resemblance of recombinant human proteins and antibodies to endogenous human proteins, protein instability remains an important concern. Handling of therapeutic antibody construct formulations are associated with significant challenges, as the conditions must maintain higher-order protein structure while minimizing degradation and aggregation, which negatively impact therapeutic effectiveness and increase potential immunogenicity to the patient. Many protein therapeutics are lyophilized to address the challenges associated with storage and transport. Handling and preparation of the therapeutic at the clinical site, however, also introduces conditions which promote instability, including formation of undesirable high molecular weight species which may elicit an unwanted immune response or adverse side effects or effect overall efficacy. The bispecific T cell engaging antigen binding polypeptides described herein, for example, have a tendency under certain conditions to form aggregates upon reconstitution of lyophilized formulations. While the level of HMW species may dissociate over time, the time period required to reach “equilibrium” (i.e., reach a steady, target level of HMW species) can be significant. For example, reconstitution of a lyophilized formulation comprising a BiTE® molecule described herein in water for injection prompts a spike in formation of HMW species which requires over 24 hours to dissociate at 8° C. and 12 hours or more at 25° C. This presents challenges at clinical sites as lyophilized formulations are reconstituted and stored for an extended time period before administration to a subject in an effort to minimize HMW species. This is inconvenient, requires additional storage space for reconstituted formulations, and adds a layer of intervention that could further affect stability. Surprisingly, reconstitution of a lyophilized formulation of the polypeptide described herein with a diluent comprising an aromatic alcohol (e.g., benzyl alcohol), a phenolic compound (e.g., phenol or m-cresol), or an amino acid with aromatic side chain (phenylalanine) significantly reduces the time required for HMW species in the reconstituted composition to dissociate. As such, the time between reconstitution and administration is reduced, providing a practical advantage to clinicians and patients.

The disclosure provides a method comprising (a) reconstituting a lyophilized formulation comprising a polypeptide, a saccharide, a surfactant, and a buffer, with a diluent comprising an aromatic alcohol (e.g., benzyl alcohol), a phenolic compound (e.g., phenol or m-cresol), or an amino acid with aromatic side chain (phenylalanine), and (b) administering the reconstituted formulation to a subject in need thereof within 12 hours of reconstitution. In various aspects, the polypeptide binds human DLL3 and humanCD3, and optionally comprises SEQ ID NO: 75 and SEQ ID NO: 9.

Various aspects of the disclosure are described below. The use of section headings is merely for the convenience of reading, and not intended to be limiting per se. The entire document is intended to be viewed as a unified disclosure, and it should be understood that all combinations of features described herein are contemplated.

### Polypeptides

In exemplary aspects, the polypeptide of the lyophilized formulation is an antibody or immunoglobulin-like construct, which includes traditional antibody formats and antigen-binding fragments thereof, as well as constructs based on antibody structural features that allow antigen binding. The term “antibody” refers to an intact antigen-binding immunoglobulin. The antibody can be an IgA, IgD, IgE, IgG, or IgM antibody, including any one of IgG1, IgG2, IgG3 or IgG4. In various aspects, an intact antibody comprises two full-length heavy chains and two full-length light chains. An antibody has a variable region and a constant region. In IgG formats, a variable region is generally about 100-110 or more amino acids, comprises three complementarity determining regions (CDRs), is primarily responsible for antigen recognition, and substantially varies among other antibodies that bind to different antigens. A variable region typically comprises at least three heavy or light chain CDRs (Kabat et al., 1991, Sequences of Proteins of Immunological Interest, Public Health Service N.I.H., Bethesda, Md.; see also Chothia and Lesk, 1987, J. Mol. Biol. 196:901-917; Chothia et al., 1989, Nature 342: 877-883), within a framework region (designated framework regions 1-4, FR1, FR2, FR3, and FR4, by Kabat et al., 1991; see also Chothia and Lesk, 1987, supra). The constant region allows the antibody to recruit cells and molecules of the immune system.

The protein or polypeptide of the lyophilized formulation is, in various aspects, “bispecific” insofar as it binds two different antigen targets. “Multispecific” constructs also are contemplated which bind more than two different antigen targets (e.g., three different antigen targets). A “bispecific” antibody construct generally comprises a first binding domain and a second binding domain, wherein the first binding domain binds to one antigen or target (e.g., the target cell surface antigen), and the second binding domain binds to another antigen or target (e.g., CD3). Accordingly, the antibody construct optionally comprises specificities for two different antigens or targets. The term “target cell surface antigen” refers to an antigenic structure expressed by a cell and which is present at the cell surface such that it is accessible for an antibody construct as described herein. It may be a protein, preferably the extracellular portion of a protein, or a carbohydrate structure, preferably a carbohydrate structure of a protein, such as a glycoprotein.

Multispecific (e.g., bispecific) antibodies and/or multispecific (e.g., bispecific) antibody constructs include, but are not limited to, traditional bispecific immunoglobulins (e.g., BslgG), IgG comprising one or more appended antigen-binding domains (e.g., the amino or carboxy termini of light or heavy chains are connected to additional antigen-binding domains, such as single domain antibodies or paired antibody variable domains (e.g., Fv or scFv)), BsAb fragments (e.g., bispecific single chain antibodies), multispecific (e.g., bispecific) fusion polypeptides (e.g., antigen binding domains fused to an effector moiety), and BsAb conjugates. See, e.g., Spiess et al., Molecular Immunology 67(2) Part A: 97-106 (2015), which describes various bispecific formats and is hereby incorporated by reference. Examples of bispecific antibody constructs include, but are not limited to, diabodies, single chain diabodies, tandem scFvs, bispecific T cell engager (BiTE®) format (a fusion protein consisting of two single-chain variable fragments (scFvs) joined by a linker), Fab₂ bispecifics, single chain Fab₂ bispecifics, as well as engineered constructs comprising full length antibodies (e.g., full length antibodies with Fc mutations that facilitate assembly of multispecific constructs). The disclosure also contemplates scFv-single domain antibodies comprising at least one single domain antibody (e.g., two single domain antibodies) and one scFv, optionally connected by linkers. See also, e.g., Chames & Baty, 2009, mAbs 1[6]:1-9; and Holliger & Hudson, 2005, Nature Biotechnology 23[9]:1126-1136; Wu et al., 2007, Nature Biotechnology 25[11]:1290-1297;Michaelson et al., 2009, mAbs 1[2]:128-141; International Patent Publication No. 2009032782 and 2006020258; Zuo et al., 2000, Protein Engineering 13[5]:361-367; U.S. Patent Application Publication No. 20020103345; Shen et al., 2006, J Biol Chem 281[16]:10706-10714; Lu et al., 2005, J Biol Chem 280[20]:19665-19672; and Kontermann, 2012 MAbs 4(2):182, all of which are expressly incorporated herein. In various aspects, the polypeptide is a BiTE® or BiTE® HLE construct.

The term “binding domain” refers to a domain which (specifically) binds to (i.e., interacts with or recognizes) a given target epitope or a given target site on a target molecule (antigen), such as, e.g., CDH19, MSLN, DLL3, FLT3, EGFRvIII, BCMA, PSMA, CD33, CD19, CD70, CLDN18.2, MUC17, or CD3. In a bispecific antibody construct, for example, the structure and function of the first binding domain (recognizing, e.g., CDH19, MSLN, DLL3, FLT3, EGFRvIII, BCMA, PSMA, CD33, CD19, CD70, CLDN18.2, or MUC17), and preferably also the structure and/or function of the second binding domain (recognizing CD3), is/are based on the structure and/or function of an antibody, e.g., of a full-length or whole immunoglobulin molecule. Alternatively, the structure and function are drawn from the variable heavy chain (VH) and/or variable light chain (VL) domains of an antibody or fragment thereof. Preferably, the first binding domain is characterized by the presence of three light chain CDRs (i.e., CDR1, CDR2 and CDR3 of the VL region) and/or three heavy chain CDRs (i.e., CDR1, CDR2 and CDR3 of the VH region). The second binding domain preferably also comprises the minimum structural requirements of an antibody which allow for the target binding. More preferably, the second binding domain comprises at least three light chain CDRs (i.e., CDR1, CDR2 and CDR3 of the VL region) and/or three heavy chain CDRs (i.e., CDR1, CDR2 and CDR3 of the VH region). In various aspects, one or more of the antigen binding domains are human or humanized or chimeric.

In some aspects, the lyophilized protein is a bispecific antibody construct (e.g., bispecific T cell engaging antigen binding polypeptide) comprising a first binding domain that binds to a tumor antigen (e.g., DLL3), a second binding domain that binds to human CD3 on the surface of a T cell, and optionally a third domain comprising, in an amino to carboxyl order, hinge-CH2 domain-CH3 domain-linker-hinge-CH2 domain-CH3 domain. In some aspects, each of the first and second binding domains comprise a VH region and a VL region.

Binding domains and variable domains (VHNL) of an antigen binding polypeptide may or may not comprise peptide linkers (spacer peptides). A peptide linker may connect one variable and/or binding domain to another variable and/or binding domain. Peptide linkers can also be used to fuse a third domain to the other domains of a bispecific T cell engaging, antigen binding polypeptide. Among suitable peptide linkers are those described in U.S. Pat. Nos. 4,751,180 and 4,935,233 or International Patent Publication No. WO 88/09344, the disclosures of which are incorporated herein by reference in their entireties. The peptide linkers can also be used to attach other domains or modules or regions (such as half-life extending domains) to a bispecific T cell engaging antigen binding polypeptide described herein.

In some aspects, the protein or polypeptide comprises a third domain comprising all or part of an “Fc” or “Fc region” or “Fc domain,” which refers to the polypeptide comprising the constant region of an antibody excluding the first constant region immunoglobulin domain. Thus, “Fc domain” refers to the last two constant region immunoglobulin domains of IgA, IgD, and IgG, the last three constant region immunoglobulin domains of IgE and IgM, and the flexible hinge N-terminal to these domains. For IgA and IgM, Fc may include the J chain. For IgG, the Fc domain comprises immunoglobulin domains Cy2 and Cy3 (Cy2 and Cy3) and the lower hinge region between Cy1 (Cy1) and Cy2 (Cy2). The bispecific T cell engaging antigen binding polypeptide is preferably an IgG antibody (which includes several subclasses, including, but not limited to IgG1, IgG2, IgG3, and IgG4). Although the boundaries of the Fc region may vary, the human IgG heavy chain Fc region is usually defined to include residues C226 or P230 to its carboxyl-terminus, wherein the numbering is according to the EU index as in Kabat. In some aspects, amino acid modifications are made to the Fc region, for example, to alter binding to one or more FcγR receptors or to the FcRn receptor.

In some embodiments, the lyophilized formulation described herein comprises a bispecific antibody construct comprising a first binding domain that binds to a target cell surface antigen and a second binding domain that binds to human CD3 on the surface of a T cell. In any of the aspects described herein, the target cell surface antigen is CDH19, MSLN, DLL3, FLT3, EGFR, EGFRvIII, BCMA, PSMA, CD33, CD19, CD70, MUC17 or CLDN18.2. The bispecific antibody construct, in various aspects, comprises a third domain comprising, in an amino to carboxyl order, hinge-CH2 domain-CH3 domain-linker-hinge-CH2 domain-CH3 domain. In some embodiments, each of the first and second binding domains comprise a VH region and a VL region. Thus, in some embodiments, the formulations described herein comprise a bispecific antibody construct which binds human CD3 and human CDH19, or human CD3 and human MSLN, or human CD3 and human DLL3, or human CD3 and human FLT3, or human CD3 and human EGFRvIII, or human CD3 and human BCMA, or human CD3 and PSMA, or human CD3 and human CD33, or human CD3 and human CD19, human CD3 and human CD70, or human CD3 and human MUC17, or human CD3 and human CLDN18.2.

In some embodiments, the first binding domain of the bispecific antibody construct comprises a set of six CDRs set forth in (a) SEQ ID NOs: 24-29, (b) SEQ ID NOs: 34-39, (c) SEQ ID NOs: 78-83, (d) SEQ ID NOs: 10-15, (e) SEQ ID NOs: 46-51, (f) SEQ ID NOs: 88-93, (g) SEQ ID NOs: 67-72, (h) SEQ ID NOs: 56-61, (i) SEQ ID NOs: 112-117, ( ) SEQ ID NOs: 100-105, (k) SEQ ID NOs:148-153, SEQ ID NOs: 157-162, or SEQ ID NOs: 166-171, or SEQ ID NOs: 175-180, (I) SEQ ID NOs:132-137, or (m) SEQ ID NOs: 123-128.

In some embodiments, the first binding domain of the bispecific antibody construct comprises a VH region comprising an amino acid sequence at least 90% identical (e.g., 91%, 92%, 93%, 94%, 95%, 96%, 97%, 98%, 99%, or 100% identical) to the amino acid sequence set forth in SEQ ID NO: 30, 40, 84, 16, 17, 52, 94, 73, 62, 118, 154, 163, 172, 181, 106, 138, 143, or 129. In some embodiments, the first binding domain of the bispecific antibody construct comprises a VH comprising the amino acid sequence set forth in SEQ ID NO: 30, 40, 84, 16, 17, 52, 94, 73, 62, 118, 154, 163,172, 181, 106, 138, 143, or 129.

In some embodiments, the first binding domain of the bispecific antibody construct comprises a VL region comprising an amino acid sequence at least 90% identical (e.g., 91%, 92%, 93%, 94%, 95%, 96%, 97%, 98%, 99%, or 100% identical) to the amino acid sequence set forth in SEQ ID NO: 31, 41, 85, 18, 19, 53, 95, 74, 63, 119, 155, 164, 173, 182, 107, 139, 144, or 130. In some embodiments, the first binding domain of the bispecific antibody construct comprises a VL comprising the amino acid sequence set forth in SEQ ID NO: 31, 41, 85, 18, 19, 53, 95, 74, 63, 119,155, 164, 173, 182, 107, 139, 144, or 130.

In some embodiments, the first binding domain comprises (a) a VH region comprising an amino acid sequence set forth in SEQ ID NO: 30 and a VL region comprising an amino acid sequence set forth in SEQ ID NO: 31; (b) a VH region comprising an amino acid sequence set forth in SEQ ID NO: 40 and a VL region comprising an amino acid sequence set forth in SEQ ID NO: 41; (c) a VH region comprising an amino acid sequence set forth in SEQ ID NO: 84 and a VL region comprising an amino acid sequence set forth in SEQ ID NO: 85; (d) a VH region comprising an amino acid sequence set forth in SEQ ID NO: 16 or 17 and a VL region comprising an amino acid sequence set forth in SEQ ID NO: 18 or 19; (e) a VH region comprising an amino acid sequence set forth in SEQ ID NO: 52 and a VL region comprising an amino acid sequence set forth in SEQ ID NO: 53; (f) a VH region comprising an amino acid sequence set forth in SEQ ID NO: 94 and a VL region comprising an amino acid sequence set forth in SEQ ID NO: 95; (g) a VH region comprising an amino acid sequence set forth in SEQ ID NO: 73 and a VL region comprising an amino acid sequence set forth in SEQ ID NO: 74; (h) a VH region comprising an amino acid sequence set forth in SEQ ID NO: 62 and a VL region comprising an amino acid sequence set forth in SEQ ID NO: 63; (i) a VH region comprising an amino acid sequence set forth in SEQ ID NO: 118 and a VL region comprising an amino acid sequence set forth in SEQ ID NO: 119; ( ) a VH region comprising an amino acid sequence set forth in SEQ ID NO: 154, 163, 172 or 181 and a VL region comprising an amino acid sequence set forth in SEQ ID NO: 155, 164, 173 or 182; (k) a VH region comprising an amino acid sequence set forth in SEQ ID NO: 106 and a VL region comprising an amino acid sequence set forth in SEQ ID NO: 107; (I) a VH region comprising an amino acid sequence set forth in SEQ ID NO: 138 or 143 and a VL region comprising an amino acid sequence set forth in SEQ ID NO: 139 or 144; or (m) a VH region comprising an amino acid sequence set forth in SEQ ID NO: 129 and a VL region comprising an amino acid sequence set forth in SEQ ID NO: 130.

In some embodiments, the second binding domain of the bispecific antibody construct comprises a set of six CDRs set forth in SEQ ID NOs: 1-6. In some embodiments, the second binding domain of the bispecific antibody construct comprises a VH region comprising an amino acid sequence at least 90% identical (e.g., 91%, 92%, 93%, 94%, 95%, 96%, 97%, 98%, 99%, or 100% identical) to the amino acid sequence set forth in SEQ ID NO: 7. In some embodiments, the second binding domain of the bispecific antibody construct comprises a VH comprising the amino acid sequence set forth in SEQ ID NO: 7. In some embodiments, the second binding domain of the bispecific antibody construct comprises a VL region comprising an amino acid sequence at least 90% identical (e.g., 91%, 92%, 93%, 94%, 95%, 96%, 97%, 98%, 99%, or 100% identical) to the amino acid sequence set forth in SEQ ID NO: 8. In some embodiments, the second binding domain of the bispecific antibody construct comprises a VL comprising the amino acid sequence set forth in SEQ ID NO: 8. In some embodiments, the second binding domain comprises a VH region comprising an amino acid sequence set forth in SEQ ID NO: 7 and a VL region comprising an amino acid sequence set forth in SEQ ID NO: 8.

In some embodiments, the bispecific antibody construct comprises a first binding domain that binds CD19 comprising an anti-CD19 variable light domain comprising the amino acid sequence of SEQ ID NO: 85 and an anti-CD19 variable heavy domain comprising the amino acid sequence of SEQ ID NO: 84, a second binding domain comprising an anti-CD3 variable heavy domain comprising the amino acid sequence of SEQ ID NO: 7, and an anti-CD3 variable light domain comprising the amino acid sequence of SEQ ID NO: 8. For example, in an embodiment, the bispecific antibody construct comprises a first binding domain comprising the amino acid sequence of SEQ ID NO: 86 and a second binding domain comprising the amino acid sequence of SEQ ID NO: 9. In some embodiments, the bispecific antibody construct comprises the amino acid sequence set forth in SEQ ID NO: 87.

In some embodiments, the bispecific antibody construct comprises a first binding domain that binds MSLN comprising an anti-MSLN variable light domain comprising the amino acid sequence of SEQ ID NO: 41 and an anti-MSLN variable heavy domain comprising the amino acid sequence of SEQ ID NO: 40, a second binding domain comprising an anti-CD3 variable heavy domain comprising the amino acid sequence of SEQ ID NO: 7, and an anti-CD3 variable light domain comprising the amino acid sequence of SEQ ID NO: 8. For example, in one embodiment, the bispecific antibody construct comprises a first binding domain comprising the amino acid sequence of SEQ ID NO: 42 and a second binding domain comprising the amino acid sequence of SEQ ID NO: 9. In some embodiments, the bispecific antibody construct comprises an amino acid sequence set forth in SEQ ID NO: 43, 44, or 45.

In some embodiments, the bispecific antibody construct comprises a first binding domain that binds FLT3 comprising an anti-FLT3 variable light domain comprising the amino acid sequence of SEQ ID NO: 63 and an anti-FLT3 variable heavy domain comprising the amino acid sequence of SEQ ID NO: 62, a second binding domain comprising an anti-CD3 variable heavy domain comprising the amino acid sequence of SEQ ID NO: 7, and an anti-CD3 variable light domain comprising the amino acid sequence of SEQ ID NO: 8. For example, in one embodiment, the bispecific antibody construct comprises a first binding domain comprising the amino acid sequence of SEQ ID NO: 64 and a second binding domain comprising the amino acid sequence of SEQ ID NO: 9. In some embodiments, the bispecific antibody construct comprises an amino acid sequence set forth in SEQ ID NO: 65 or 66.

In some embodiments, the bispecific antibody construct comprises a first binding domain that binds EGFRvII comprising an anti-EGFRvIII variable light domain comprising the amino acid sequence of SEQ ID NO: 31 and an anti-EGFRvIII variable heavy domain comprising the amino acid sequence of SEQ ID NO: 30, a second binding domain comprising an anti-CD3 variable heavy domain comprising the amino acid sequence of SEQ ID NO: 7, and an anti-CD3 variable light domain comprising the amino acid sequence of SEQ ID NO: 8. For example, in an embodiment, the bispecific antibody construct comprises a first binding domain comprising the amino acid sequence of SEQ ID NO: 32 and a second binding domain comprising the amino acid sequence of SEQ ID NO: 9. In some embodiments, the bispecific antibody construct comprises an amino acid sequence set forth in SEQ ID NO: 33.

In some embodiments, the bispecific antibody construct comprises a first binding domain that binds BCMA comprising an anti-BCMA variable light domain comprising the amino acid sequence of SEQ ID NO: 95 and an anti-BCMA variable heavy domain comprising the amino acid sequence of SEQ ID NO: 94, a second binding domain comprising an anti-CD3 variable heavy domain comprising the amino acid sequence of SEQ ID NO: 7, and an anti-CD3 variable light domain comprising the amino acid sequence of SEQ ID NO: 8. For example, in an embodiment, the bispecific antibody construct comprises a first binding domain comprising the amino acid sequence of SEQ ID NO: 96 and a second binding domain comprising the amino acid sequence of SEQ ID NO: 9. In some embodiments, the bispecific antibody construct comprises an amino acid sequence set forth in SEQ ID NO: 98 or SEQ ID NO: 97.

In some embodiments, the bispecific antibody construct comprises a first binding domain that binds PSMA comprising an anti-PSMA variable light domain comprising the amino acid sequence of SEQ ID NO: 119 or 107 and an anti-PSMA variable heavy domain comprising the amino acid sequence of SEQ ID NO: 118 or 106, a second binding domain comprising an anti-CD3 variable heavy domain comprising the amino acid sequence of SEQ ID NO: 7, and an anti-CD3 variable light domain comprising the amino acid sequence of SEQ ID NO: 8. For example, in an embodiment, the bispecific antibody construct comprises a first binding domain comprising the amino acid sequence of SEQ ID NO: 120 or 108 and a second binding domain comprising the amino acid sequence of SEQ ID NO: 9. In some embodiments, the bispecific antibody construct comprises an amino acid sequence set forth in SEQ ID NO: 121, 122, 109, 110, or 111.

In some embodiments, the bispecific antibody construct comprises a first binding domain that binds CD33 comprising an anti-CD33 variable light domain comprising the amino acid sequence of SEQ ID NO: 18 or 19 and an anti-CD33 variable heavy domain comprising the amino acid sequence of SEQ ID NO: 16 or 17, a second binding domain comprising an anti-CD3 variable heavy domain comprising the amino acid sequence of SEQ ID NO: 7, and an anti-CD3 variable light domain comprising the amino acid sequence of SEQ ID NO: 8. For example, in one embodiment, the bispecific antibody construct comprises a first binding domain comprising the amino acid sequence of SEQ ID NO: 189 or 190 and a second binding domain comprising the amino acid sequence of SEQ ID NO: 9. In some embodiments, the bispecific antibody construct comprises the amino acid sequence set forth in SEQ ID NO: 20, 21, 22, or 23.

In some embodiments, the bispecific antibody construct comprises a first binding domain that binds CDH19 comprising an anti-CDH19 variable light domain comprising the amino acid sequence of SEQ ID NO: 53 and an anti-CDH19 variable heavy domain comprising the amino acid sequence of SEQ ID NO: 52, a second binding domain comprising an anti-CD3 variable heavy domain comprising the amino acid sequence of SEQ ID NO: 7, and an anti-CD3 variable light domain comprising the amino acid sequence of SEQ ID NO: 8. For example, in one embodiment, the bispecific antibody construct comprises a first binding domain comprising the amino acid sequence of SEQ ID NO: 54 and a second binding domain comprising the amino acid sequence of SEQ ID NO: 9. In some embodiments, the bispecific antibody construct comprises the amino acid sequence set forth in SEQ ID NO: 55.

In some embodiments, the bispecific antibody construct comprises a first binding domain that binds MUC17 comprising an anti-MUC17 variable light domain comprising the amino acid sequence of SEQ ID NO: 155, 164, 173, or 182 and an anti-MUC17 variable heavy domain comprising the amino acid sequence of SEQ ID NO: 154, 163, 172, or 181, a second binding domain comprising an anti-CD3 variable heavy domain comprising the amino acid sequence of SEQ ID NO: 7, and an anti-CD3 variable light domain comprising the amino acid sequence of SEQ ID NO: 8. For example, in embodiments, the bispecific antibody construct comprises a first binding domain comprising the amino acid sequence of SEQ ID NO: 194 and a second binding domain comprising the amino acid sequence set forth in SEQ ID NO: 195 (optionally with an Fc domain comprising the amino acid sequence of SEQ ID NO: 196). In some embodiments, the bispecific antibody construct comprises the amino acid sequence set forth in SEQ ID NO: 156, 165, 174, or 183.

In some embodiments, the bispecific antibody construct comprises a first binding domain that binds cldn18.2 comprising an anti-cldn18.2 variable light domain comprising the amino acid sequence of SEQ ID NO: 139 or 144 and an anti-cldn18.2 variable heavy domain comprising the amino acid sequence of SEQ ID NO: 138 or 143, a second binding domain comprising an anti-CD3 variable heavy domain comprising the amino acid sequence of SEQ ID NO: 7, and an anti-CD3 variable light domain comprising the amino acid sequence of SEQ ID NO: 8. For example, in one embodiment, the bispecific antibody construct comprises a first binding domain comprising the amino acid sequence of SEQ ID NO: 140 or 145 and a second binding domain comprising the amino acid sequence of SEQ ID NO: 9. In some embodiments, the bispecific antibody construct comprises the amino acid sequence set forth in SEQ ID NO: 141, 142, 146, or 147.

In some embodiments, the bispecific antibody construct comprises a first binding domain that binds CD70 comprising an anti-CD70 variable light domain comprising the amino acid sequence of SEQ ID NO: 130 and an anti-CD70 variable heavy domain comprising the amino acid sequence of SEQ ID NO: 129, a second binding domain comprising an anti-CD3 variable heavy domain comprising the amino acid sequence of SEQ ID NO: 7, and an anti-CD3 variable light domain comprising the amino acid sequence of SEQ ID NO: 8. For example, in embodiments, the bispecific antibody construct comprises a first binding domain comprising the amino acid sequence of SEQ ID NO: 191 and a second binding domain comprising the amino acid sequence set forth in SEQ ID NO: 192 (optionally with an Fc domain comprising the amino acid sequence of SEQ ID NO: 193). In some embodiments, the bispecific antibody construct comprises an amino acid sequence set forth in SEQ ID NO: 131.

In various aspects, the lyophilized formulation comprises a polypeptide that binds to human DLL3 (Delta-like ligand 3; UniProtKB-Q9NYJ7) and human CD3 (Cluster of differentiation 3). In this regard, the polypeptide is an antigen-binding protein or polypeptide comprising a scaffold or framework portion that allows an antigen binding domain to adopt a conformation that promotes binding of the antigen binding polypeptide to the antigen (i.e., DLL3 or CD3). In various aspects of the disclosure, the polypeptide comprises antigen binding domains that comprise the amino acid sequences of SEQ ID NOs: 75 and 9. The structure and function of antigen binding domains may be based on the structure and/or function of an antibody, e.g., of a full-length or whole immunoglobulin molecule and/or drawn from the variable heavy chain (VH) and/or variable light chain (VL) domains of an antibody. Binding domains are typically characterized by the presence of three light chain CDRs (i.e., CDR1, CDR2 and CDR3 of the VL region) and/or three heavy chain CDRs (i.e., CDR1, CDR2 and CDR3 of the VH region) alongside minimum structural requirements which allow for the target binding. In some aspects, the binding domain comprises a single chain antigen binding polypeptide comprising, e.g., a variable heavy chain, a scFv linker, and a variable light domain. Optionally, the C-terminus of a variable light chain is attached to the N-terminus of the scFv linker, the C-terminus of which is attached to the N-terminus of a variable heavy chain (N-vh-linker-vl-C), although the configuration can be switched (N-vl-linker-vh-C). Alternatively, the C-terminus of the variable heavy chain may be attached to the N-terminus of the scFv linker, the C-terminus of which is attached to the N-terminus of a variable light chain (N-vl-linker-vh-C), although the configuration can be switched (N-vh-linker-v-C).

The formulation described herein, in various aspects, comprises a polypeptide (e.g., a bispecific T cell engaging antigen binding polypeptide) which binds human CD3 and human DLL3. In some aspects, the polypeptide comprises a first binding domain that binds DLL3 and comprises a set of six CDRs set forth in SEQ ID NOs: 67-72. In some aspects, the DLL3 binding domain comprises a VH region comprising an amino acid sequence at least 90% identical (e.g., 91%, 92%, 93%, 94%, 95%, 96%, 97%, 98%, 99%, or 100% identical) to the amino acid sequence set forth in SEQ ID NO: 73. In some aspects, the DLL3 binding domain of the polypeptide comprises a VL region comprising an amino acid sequence at least 90% identical (e.g., 91%, 92%, 93%, 94%, 95%, 96%, 97%, 98%, 99%, or 100% identical) to the amino acid sequence set forth in SEQ ID NO: 74. For example, the DLL3 binding domain may comprise a VH region comprising an amino acid sequence set forth in SEQ ID NO: 73 and a VL region comprising an amino acid sequence set forth in SEQ ID NO: 74, such as a binding domain comprising the amino acid of SEQ ID NO: 75. In some aspects, the CD3 binding domain of the polypeptide comprises a set of six CDRs set forth in SEQ ID NOs: 1-6. For example, the CD3 binding domain optionally comprises a VH region comprising an amino acid sequence at least 90% identical (e.g., 91%, 92%, 93%, 94%, 95%, 96%, 97%, 98%, 99%, or 100% identical) to the amino acid sequence set forth in SEQ ID NO: 7. In some aspects, the CD3 binding domain of the polypeptide comprises a VL region comprising an amino acid sequence at least 90% identical (e.g., 91%, 92%, 93%, 94%, 95%, 96%, 97%, 98%, 99%, or 100% identical) to the amino acid sequence set forth in SEQ ID NO: 8. In some aspects, wherein the second binding domain comprises a VH region comprising an amino acid sequence set forth in SEQ ID NO: 7 and a VL region comprising an amino acid sequence set forth in SEQ ID NO: 8, such as a binding domain comprising the amino acid sequence of SEQ ID NO: 9. In some aspects, the polypeptide comprises an amino acid sequence set forth in SEQ ID NO: 76 or 77.

In various embodiments, the bispecific T cell engaging antigen binding polypeptide is tarlatamab (International Nonproprietary Names for Pharmaceutical Substances (INN): Proposed INN: List 123, WHO Drug Information 34(2): 395-397 (2020)), also known as AMG 757. Tarlatamab is an immunoglobulin scFv-scFv-scFc, anti-[Homo sapiens DLL3 (delta-like ligand 3)] and anti-[Homo sapiens CD3E (CD3 epsilon, Leu-4)], monoclonal antibody single chain (scFv)2-scFc, bispecific; IG single chain scFv-scFv-scFc, anti-DLL3 and anti-CD3E (1-982) [scFv-VH-V-kappa anti-DLL3 (1-241) [VH (Homo sapiens IGHV4-59*01 G49>C (44) (96.9%)-(IGHD)-lGHJ4*01 (100%)) CDR-IMGT [8.7.12] (26-33.51-57.96-107) (1-118)-15-mertris(tetraglycyl-seryl) linker (119-133)-V-KAPPA (Homo sapiens IGKV3-20*01 (91.7%)-lGKJ2*01 Q120>C (234) (90.9%)) CDRIMGT [7.3.9] (160-166.184-186.223-231) (134-241)]-6-merseryl-tetraglycyl-seryl linker (242-247)-scFv-VH-V-lambda antiCD3E (248-496) [VH (Mus musculuslGHV10-1*02 (91.9%) -(IGHD)-lGHJ3*01 (86.7%)/Homo sapiens IGHV3-73*01 (87.0%)-(IGHD)-IGHJ5*01 (100%)) CDR-IMGT [8.10.16] (273-280.298-307.346-361) (248-372)-15-mer-tris(tetraglycyl-seryl) linker (373-387)-V-LAMBDA (Homo sapiens IGLV7-43*01 (85.1%)-lGLJ3*02 (100%)) CDR-IMGT [9.3.9] (413-421.439-441.478-486) (388-496)]-4-mer-tetraglycyl linker (497-500) -scFc (h-CH2-CH3)-(h-CH2-CH3) (501-982) [Homo sapiens IGHG1*03 h-CH2-CH3, nG1m1 (hinge 6-15 (501-510), CH2 R83>C (572), N84.4>G (577), V85>C (582) (511-620), CH3 E12 (636), M14 (638) (621-725), CHS>del) (501-725)-30-merhexakis(tetraglycyl-seryl) linker (726-755)-Homo sapiens IGHG1*03 h-CH2-CH3, nG1m1 (hinge 6-15 (756-765), CH2 R83>C (827), N84.4>G (832), V85>C (837) (766-875), CH3 E12 (891), M14 (893) (876-980), CHS (981-982)) (756-982), non-glycosylated, produced in Chinese hamster ovary (CHO) cells; immunomodulator, antineoplastic.

Optionally, the polypeptide comprises the amino acid sequence set forth in SEQ ID NO: 20, SEQ ID NO: 21, SEQ ID NO: 22, SEQ ID NO: 23, SEQ ID NO: 33, SEQ ID NO: 43, SEQ ID NO: 44, SEQ ID NO: 45, SEQ ID NO: 55, SEQ ID NO: 65, SEQ ID NO: 66, SEQ ID NO: 76, SEQ ID NO: 77, SEQ ID NO: 87, SEQ ID NO: 97, SEQ ID NO: 98, SEQ ID NO: 99, SEQ ID NO: 109, SEQ ID NO: 110, SEQ ID NO: 111, SEQ ID NO: 121, SEQ ID NO: 122, SEQ ID NO: 131, SEQ ID NO: 141, SEQ ID NO: 142, SEQ ID NO: 146, SEQ ID NO: 147, SEQ ID NO: 156, SEQ ID NO: 165, SEQ ID NO: 174, SEQ ID NO: 183, SEQ ID NO: 184, SEQ ID NO: 185, SEQ ID NO: 186, SEQ ID NO: 187, or SEQ ID NO: 188, such as the amino acid sequence set forth in SEQ ID NO: 76, SEQ ID NO: 77, SEQ ID NO: 97, SEQ ID NO: 98, SEQ ID NO: 99, SEQ ID NO: 156, SEQ ID NO: 165, SEQ ID NO: 174, or SEQ ID NO: 183.

In some aspects, the reconstituted formulation comprises the polypeptide (e.g., the bispecific T cell engaging antigen binding polypeptide) in a concentration ranging from about 0.5 mg/mL to about 50 mg/mL (e.g., from about 1 mg/mL to about 20 mg/mL or about 15 mg/mL). In some aspects, the reconstituted formulation comprises the polypeptide at a concentration of about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, or about 20 mg/mL, or any range between these endpoints. Alternatively, the reconstituted formulation comprises the polypeptide at a concentration of about 25 mg/mL, about 30 mg/mL, about 35 mg/mL, about 40 mg/mL, about 45 mg/mL, or about 50 mg/mL, or any range between these endpoints. In various aspects, the reconstituted formulation comprises the polypeptide at a concentration of at least about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, or about 10 mg/mL, and no higher than about 15 mg/mL, 20 mg/mL, 25 mg/mL, about 30 mg/mL, about 35 mg/mL, about 40 mg/mL, about 45 mg/mL, or about 50 mg/mL. For example, the reconstituted formulation optionally comprises the polypeptide in a concentration of about 15 mg/mL.

### Buffers

The formulation of the disclosure comprises a buffer, which optionally is an acetate buffer, a glutamate buffer, a citrate buffer, a succinate buffer, a tartrate buffer, a fumarate buffer, a maleate buffer, a histidine buffer, a phosphate buffer, or a combination thereof. Optionally, the buffer is a glutamate buffer.

In some aspects, the buffer is present in the formulation at concentration that maintains a pH of about 4 to about 7, about 4 to about 6, about 4 to about 5, or about 4.2. The effect of pH on formulations may be characterized using any one or more of several approaches such as accelerated stability studies and calorimetric screening studies (Remmele R. L. Jr., et al., Biochemistry, 38(16): 5241-7 (1999)). In the context of the lyophilized formulation, the buffer may be present in the pre-lyophilized formulation at a concentration between about 0.1 mM and about 1000 mM (1 M), such as between about 5 mM and about 200 mM (e.g., between about 5 mM to about 100 mM). By “pre-lyophilized formulation” is meant the formulation just prior to lyophilization. In various aspects, the buffer is present in the pre-lyophilized formulation at a concentration between about 10 mM and 50 about mM. Suitable buffer concentrations encompass concentrations of about 200 mM or less. In some aspects, the concentration of the buffer is (i) at least about 1 mM, at least about 2 mM, at least about 3 mM, at least about 4 mM, at least about 5 mM, at least about 6 mM, at least about 7 mM, at least about 8 mM, at least about 9 mM, at least about 10 mM, at least about 11 mM, at least about 12 mM, at least about 13 mM, at least about 14 mM, or at least about 15 mM, and (ii) no more than about 20 mM, no more than about 30 mM, no more than about 40 mM, no more than about 50 mM, no more than about 60 mM, no more than about 70 mM, no more than about 80 mM, no more than about 90 mM, no more than about 100 mM, or no more than about 200 mM. In some aspects, the buffer in the in the pre-lyophilized formulation is present in a concentration of about 190 mM, about 180 mM, about 170 mM, about 160 mM, about 150 mM, about 140 mM, about 130 mM, about 120 mM, about 110 mM, about 100 mM, about 80 mM, about 70 mM, about 60 mM, about 50 mM, about 40 mM, about 30 mM, about 20 mM, about 19 mM, about 18 mM, about 17 mM, about 16 mM, about 15 mM, about 14 mM, about 13 mM, about 12 mM, about 11 mM, about 10 mM or about 5 mM. In some aspects, the concentration of the buffer (e.g., glutamate) in the pre-lyophilized formulation is about 15 mM.

### Surfactants

The formulations described here comprise a surfactant. Exemplary surfactants include, but are not limited to, polysorbate 20, polysorbate 40, polysorbate 60, polysorbate 80, poloxamer 188, poloxamer 407, Triton® X-100 (2-[4-(2,4,4-trimethylpentan-2-yl)phenoxy]ethanol; CAS #9002-93-1), polyoxyethylene, PEG 3350, and PEG 4000, as well as combinations thereof. Optionally, the buffer is polysorbate 80.

In some aspects, the pre-lyophilized formulation comprises a surfactant at a concentration of about 0.001% to about 5% w/v. For example, the concentration is, in various instances, about 0.001% to about 0.5% (such as about 0.004 to about 0.5% w/v, or about 0.001 to about 0.01% w/v, or about 0.004 to about 0.01% w/v). In some aspects, the pre-lyophilized formulation comprises a surfactant at a concentration of (i) at least about 0.001%, at least about 0.002%, at least about 0.003%, at least about 0.004%, at least about 0.005%, at least about 0.007%, at least about 0.01%, at least about 0.05%, at least about 0.1%, at least about 0.2%, at least about 0.3%, at least about 0.4%, at least about 0.5%, at least about 0.6%, at least about 0.7%, at least about 0.8%, at least about 0.9%, or at least about 1.0% w/v, and (ii) no greater than about 2.0%, no greater than about 1.9% no greater than about 1.8%, no greater than about 1.7%, no greater than about 1.6%, no greater than about 1.5%, no greater than about 1.4%, no greater than about 1.3%, no greater than about 1.2%, no greater than about 1.1%, no greater than about 1.0%, no greater than about 0.9%, no greater than about 0.8%, no greater than about 0.7%, no greater than about 0.6%, or no greater than about 0.5% w/v. In some aspects, the formulation comprises a surfactant incorporated in a concentration of about 0.005% to about 0.01% w/v, such as 0.01% w/v. In some aspects, the surfactant is polysorbate 80 and the polysorbate 80 is present in a concentration of about 0.01% w/v.

### Saccharides

The formulation described herein comprises a saccharide. In some aspects of the disclosure, the saccharide is a monosaccharide or a disaccharide. For example, the saccharide is optionally glucose, galactose, fructose, xylose, sucrose, lactose, maltose, trehalose, sorbitol, mannitol, or xylitol, or a combination thereof. In some aspects, the saccharide is sucrose.

In some aspects, the formulation comprises a saccharide at a concentration of about 0.01% to about 20% w/v, or about 1% to about 15%, or about 5% to about 12%, or about 7% to about 12% w/v, pre-lyophilization. In some aspects, the formulation comprises at least one saccharide at a concentration of (i) at least about 0.5%, at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, or at least about 15% w/v, and (ii) no greater than about 20%, no greater than about 18%, no greater than about 16%, no greater than about 15%, no greater than about 14%, no greater than about 13%, no greater than about 12%, no greater than about 11%, no greater than about 10%, no greater than about 9%, no greater than about 8%, no greater than about 7%, no greater than about 6%, or no greater than about 5% w/v, pre-lyophilization. In a yet further aspect, the formulation comprises at least one saccharide at a concentration of about 7%, about 7.5%, about 8%, about 8.5%, about 9%, about 9.5%, about 10%, about 10.5%, about 11%, about 11.5%, or about 12% w/v. In some aspects, the at least one saccharide is in the formulation at a concentration of about 9% w/v. In some aspects, the saccharide is sucrose and is present in the formulation ranging from about 7% to about 10% w/v, such as 9%.

Optionally, the formulation, prior to lyophilization, comprises 15 mM glutamate, 9% (wN) sucrose, and 0.01% (wN) polysorbate 80. The pH of the formulation is optionally about 4.2.

### Aggregation/High Molecular Weight Species

The term “aggregation” used herein refers to the direct mutual attraction between molecules, e.g., via van der Waals forces or chemical bonding. In particular, aggregation is understood as proteins accumulating and clumping together. Aggregates may include amorphous aggregates and oligomers and are typically referred to as high molecular weight (HMW) species, i.e., molecules having a higher molecular weight than product molecules which are non-aggregated molecules. Protein aggregates (HMW species) may generally differ in size (ranging from small (dimers) to large assemblies (subvisible or even visible particles) and from the nanometer to micrometer range in diameter), morphology (approximately spherical to fibrillar), protein structure (native vs. non-native/denatured), type of intermolecular bonding (covalent vs. non-covalent), reversibility, and solubility. Soluble aggregates cover the size range of roughly 1 to 100 nm, and protein particulates cover subvisible (˜0.1-100 nm) and visible (>100 nm) ranges.

The stability of a protein formulation can be quantified in several ways. In some aspects, stability of a polypeptide formulation (e.g., a bispecific T cell engaging antigen binding polypeptide formulation) is characterized by size exclusion high performance liquid chromatography (SE-HPLC), size exclusion ultra high performance liquid chromatography (SE-UHPLC), cation exchange high performance liquid chromatography (CE-HPLC), dynamic light scattering, analytical ultracentrifugation (AUC), field flow fractionation (FFF), isoelectric focusing and ion exchange chromatography (IEX). In some aspects, stability of a polypeptide formulation is characterized by partial dissociation as measured by sodium-dodecyl sulfate capillary electrophoresis (CE-SDS) and/or sodium-dodecyl sulfate polyacrylamide gel electrophoresis (SDS-PAGE). In some aspects, stability of the formulation is assessed by reduced capillary electrophoresis-sodium dodecyl sulfate (rCE-SDS).

Stability of formulations may be characterized by the amount of high molecular weight (HMW) species of a polypeptide (e.g., bispecific T cell engaging antigen binding polypeptide) or by the rate of increase of the amount of HMW species of the polypeptide (e.g., bispecific T cell engaging antigen binding polypeptide) under predetermined temperatures and at various time points (e.g., various time points after reconstitution of the lyophilized formulation). For example, the amount of HMW species may be determined at one hour, two hours, six hours, 10 hours, 12 hours, 15 hours, 24 hours, 25 hours, or more at approximately 8° C. or 25° C. In some aspects, the rate of decrease of HMW species is determined at one hour, two hours, six hours, 12 hours, 24 hours, 25 hours or more at approximately 8° C. or 25° C. In some aspects, the HMW species of a bispecific T cell engaging, antigen binding polypeptide in the formulation is measured by SE-UHPLC.

In some aspects, the relative values of any particular species of the polypeptide (e.g., bispecific T cell engaging, antigen binding polypeptide), as described herein, such as the intact BiTE® molecule or main species, or the high molecular weight (HMW) species (i.e., aggregates) are expressed in relation to the respective values of the total product. For example, in some aspects, the reconstitution formulation comprises 4% or less, 3.5% or less, 3% or less, or 2.5% or less (e.g., 3.5%, or 3%, 2.5%, or 2%, or 1.9%, or 1.8%, or 1.7%, or 1.6%, or 1.5%, or 1.4%, or 1.3%, or 1.2%, or 1.1%, or 1%, or 0.5%) HMW species of the bispecific T cell engaging antigen binding polypeptide. In various aspects, the reconstitution formulation comprises 3% or less HMW species of the bispecific T cell engaging antigen binding polypeptide. In some aspects, the HMW species of a polypeptide (e.g., bispecific T cell engaging antigen binding polypeptide) in the formulation is measured by SE-UHPLC. Optionally, the reconstitution formulation comprises the amount of HMW species referenced above at 25 hours when stored at 8° C. or at 12 hours when stored at 25° C.

### Reconstitution and Administration

The method of the disclosure comprises reconstituting the lyophilized formulation with a diluent comprising an aromatic alcohol, a phenolic compound, or an amino acid with aromatic side chain, and administering the reconstituted formulation to a subject in need thereof within 12 hours of reconstitution. Surprisingly, reconstituting the lyophilized formation described herein with a diluent comprising a benzene-ring containing compound, such as an aromatic alcohol (e.g., benzyl alcohol), a phenolic compound (e.g., phenol or m-Cresol), or an amino acid with aromatic side chain (e.g., phenylalanine), significantly reduces the time required for HMW species to dissipate to target levels, thereby allowing administration of the reconstituted formulation to a subject much sooner after reconstitution than previously believed. In various aspects, the diluent comprises benzyl alcohol, phenol, m-Cresol, or phenylalanine, or a combination of any of the foregoing. Alternatively or in addition, the diluent may comprise benzoic acid, sodium benzoate, thimerosal, phenoxyethanol, methylparaben, phenoxyethanol, propylparaben, tyrosine, or tryptophan, or a combination of any of the foregoing. Alternatively or in addition, the diluent includes NaCl and benzyl alcohol (e.g., 0.9% benzyl alcohol) or IVSS (intravenous solution stabilizer). Optionally, the diluent comprises 0.9% benzyl alcohol, 0.8% phenol, or 0.9% m-Cresol. Optionally, the diluent comprises about 1 mM to about 100 mM of any of the diluent compositions described here (e.g., benzyl alcohol, phenol, phenylalanine, or m-Cresol). For example, in various aspects, the diluent comprises at least about 40 mM benzyl alcohol (e.g., at least about 45 mM benzyl alcohol, or at least about 50 mM benzyl alcohol). Any suitable vehicle may be used in the diluent, such as sterile water or sterile water-for-injection. The vehicle optionally does not comprise saline.

In various aspects, the method comprises administering the reconstituted formulation to a subject within 12 hours, within ten hours, within nine hours, within eight hours, within seven hours, within six hours, within five hours, within four hours, within three hours, within two hours, or within one hour of reconstitution. In this context, “reconstitution” means dissolution of the lyophilized cake into a solution. In various aspects, the method comprises administering the reconstituted formulation to the subject within 60 minutes, within 45 minutes, within 30 minutes, or within 15 minutes of reconstitution.

In some instances, the reconstituted formulation is further diluted for administration to a subject in need thereof. For example, the reconstituted formulation may be admixed with other carriers in an i.v. bag (i.e., also referred to herein as “a systemic delivery system”) in order to provide an increased dose to a subject. In this regard, the reconstituted formulation is optionally incorporated into a systemic delivery system within twelve hours, within 12 hours, within ten hours, within nine hours, within eight hours, within seven hours, within six hours, within five hours, within four hours, within three hours, within two hours, or within one hour of reconstitution. In various aspects, the method comprises incorporating the reconstituted formulation into a systemic delivery system within 60 minutes, within 45 minutes, within 30 minutes, or within 15 minutes of reconstitution.

Preferably, the formulation is administered parenterally, e.g., intravenously, subcutaneously, or intramuscularly. Parenteral administration may be achieved by injection, such as bolus injection, or by infusion, such as continuous infusion. Administration may be achieved via depot for long-term release. In some aspects, the formulation is administered intravenously by an initial bolus followed by a continuous infusion to maintain therapeutic circulating levels of drug product. In some aspects, the formulation is administered as a one-time dose. Examples of medical devices for administering formulations are described in U.S. Pat. Nos. 4,475,196; 4,439,196; 4,447,224; 4,447, 233; 4,486,194; 4,487,603; 4,596,556; 4,790,824; 4,941,880; 5,064,413; 5,312,335; 5,312,335; 5,383,851; and 5,399,163.

### Use

The formulation described herein is useful as a pharmaceutical formulation in the treatment of cancer in a subject in need thereof. The terms “subject in need” or those “in need of treatment” includes those suffering from the disorder, as well as those in which the disorder has not yet clinically manifested. The “subject” or “patient” includes human and other mammalian subjects that receive either prophylactic or therapeutic treatment. Mammalian subjects include humans, non-human primates, dogs, cats, guinea pigs, rabbits, rats, mice, horses, cattle, cows, and the like, with humans being preferred. The reconstituted formulation of the disclosure is pharmaceutically acceptable, i.e., capable of eliciting the desired therapeutic effect without causing significant undesirable local or systemic effects in the subject to which the pharmaceutical formulation is administered. The term “pharmaceutically acceptable” can mean approved by a regulatory agency or other generally recognized pharmacopoeia for use in animals, and more particularly in humans, but is not limited to those approved by a regulatory agency.

Optionally, the subject is suffering from cancer (e.g., a tumor). In various aspects, the cancer overexpresses DLL3 on the surface of the tumor or cancer cells compared to normal cells. Examples of tumors or cancers include, but are not limited to, lung cancer (preferably SCLC), breast cancer, cervical cancer, colon cancer, colorectal cancer, endometrial cancer, head and neck cancer, liver cancer, ovarian cancer, pancreatic cancer, prostate cancer, skin cancer, gastric cancer, testis cancer, thyroid cancer, adrenal cancer, renal cancer, bladder cancer, uterine cancer, esophageal cancer, urothelial cancer, brain tumor or cancer, lymphoma, carcinoma, and sarcoma, and metastatic cancer diseases derived from any of the foregoing. In various aspects, the tumor or cancer is small cell lung cancer (SCLC), non-small cell lung cancer (NSCLC), glioma, glioblastoma, melanoma, neuroendocrine prostate cancer, neuroendocrine pancreatic cancer, hepatoblastoma, hepatocellular carcinoma, or a metastatic cancer disease derived from any of the foregoing. Optionally, the tumor or cancer is SCLC, NSCLC, glioma, glioblastoma, or neuroendocrine prostate cancer.

The disclosure provides a method of treating cancer (such as any of the cancers described above), comprising administering to a subject in need thereof a therapeutically effective amount of the reconstituted formulation within the time frame post-reconstitution provided herein. The term “treatment” refers to both therapeutic treatment and prophylactic or preventative measures (e.g., to cure, heal, alleviate, relieve, alter, remedy, ameliorate, improve, or affect the disease, a symptom of the disease, or a predisposition toward the disease). “Treatment” encompasses any improvement of the disease state of a patient having a tumor or cancer (e.g., metastatic cancer) by the administration of the reconstituted formulation described herein. Such an improvement may also be seen as a slowing or stopping of the progression of the tumor or cancer (e.g., metastatic cancer) of the patient. The term “prevention” as used herein means the avoidance of the occurrence or re-occurrence of a patient having a tumor or cancer (e.g., metastatic cancer) by the administration of the reconstituted formulation to a subject in need thereof.

Treatment of cancer may be determined by any of a number of ways. Any improvement in the subject's wellbeing is contemplated (e.g., at least or about a 10% reduction, at least or about a 20% reduction, at least or about a 30% reduction, at least or about a 40% reduction, at least or about a 50% reduction, at least or about a 60% reduction, at least or about a 70% reduction, at least or about an 80% reduction, at least or about a 90% reduction, or at least or about a 95% reduction of any parameter described herein). For example, a therapeutic response would refer to one or more of the following improvements in the disease: (1) a reduction in the number of neoplastic cells; (2) an increase in neoplastic cell death; (3) inhibition of neoplastic cell survival; (5) inhibition (i.e., slowing to some extent, preferably halting) of tumor growth or appearance of new lesions; (6) decrease in tumor size or burden; (7) absence of clinically detectable disease, (8) decrease in levels of cancer markers; (9) an increased patient survival rate; and/or (10) some relief from one or more symptoms associated with the disease or condition (e.g., pain). For example, the efficacy of treatment may be determined by detecting a change in tumor mass and/or volume after treatment. The size of a tumor may be compared to the initial size and dimensions as measured by CT, PET, mammogram, ultrasound, or palpation, as well as by caliper measurement or pathological examination of the tumor after biopsy or surgical resection. Response may be characterized quantitatively using, e.g., percentage change in tumor volume (e.g., the method of the disclosure results in a reduction of tumor volume by at least 10%, at least 20%, at least 30%, at least 40%, at least 50%, at least 60%, at least 70%, at least 80%, or at least 90%). In various aspects, the methods of the disclosure further comprise monitoring treatment in the subject.

### Kits

As an additional aspect, described herein are kits which comprise one or more formulations described herein packaged in a manner which facilitates reconstitution and/or administration to subjects. For example, such a kit includes the lyophilized formulation described herein, packaged in a container such as a sealed bottle, vessel, single-use or multi-use vial, prefilled syringe, or prefilled injection device, with a reconstitution diluent as described above. A suitable vehicle or carrier may be provided, such as water for injection, physiological saline solution or artificial cerebrospinal fluid, possibly supplemented with other materials common in formulations for parenteral administration. In one aspect, the formulation is packaged in a unit dosage form. The kit may further include a device suitable for administering the formulation according to a specific route of administration. Preferably, the kit further contains a label that describes use of the polypeptide described herein or formulation described herein.

### Aspects of the Disclosure

Various representative aspects of the disclosure are provided below:
Aspect 1. A method comprising (a) reconstituting a lyophilized formulation comprising a polypeptide, a saccharide, a surfactant, and a buffer, with a diluent comprising an aromatic alcohol, a phenolic compound, or an amino acid with aromatic side chain, and (b) administering the reconstituted formulation to a subject in need thereof within 12 hours of reconstitution, wherein the polypeptide binds human DLL3 and human CD3.
Aspect 2. The method of Aspect 1, wherein the diluent comprises benzyl alcohol, phenol, m-Cresol, or phenylalanine.
Aspect 3. The method of Aspect 1 or Aspect 2, wherein the pH of the reconstituted formulation is from about 4 to about 7.
Aspect 4. The method of any one of Aspects 1-3, wherein the formulation has a pH of 4.2.
Aspect 5. The method of any one of Aspects 1-4, wherein the reconstituted formulation comprises about 1 mg/mL to about 20 mg/mL of the polypeptide.
Aspect 6. The method of any one of Aspects 1-5, wherein the reconstituted formulation comprises about 15 mg/mL of the polypeptide.
Aspect 7. The method of any one of Aspects 1-6, wherein the buffer is an acetate buffer, a glutamate buffer, a citrate buffer, a lactic buffer, a succinate buffer, a tartrate buffer, a fumarate buffer, a maleate buffer, a histidine buffer, or a phosphate buffer.
Aspect 8. The method of Aspect 7, wherein the buffer is a glutamate buffer.
Aspect 9. The method of any one of Aspects 1-8, wherein the buffer is present in at a concentration ranging from 5 to 200 mM prior to lyophilization.
Aspect 10. The method of Aspect 9, wherein the buffer is present at a concentration ranging from 10 to 50 mM prior to lyophilization.
Aspect 11. The method of any one of Aspects 1-10, wherein the saccharide is a monosaccharide or a disaccharide.
Aspect 12. The method of any one of Aspects 1-11, wherein the saccharide is glucose, galactose, fructose, xylose, sucrose, lactose, maltose, trehalose, sorbitol, mannitol or xylitol.
Aspect 13. The method of Aspect 12, wherein the saccharide is sucrose.
Aspect 14. The method of any one of Aspects 1-13, wherein the saccharide is present in a concentration ranging from 1 to 15% (w/v) prior to lyophilization.
Aspect 15. The method of any one of Aspects 1-14 wherein the surfactant is polysorbate 20, polysorbate 40, polysorbate 60, polysorbate 80, poloxamer 188, poloxamer 407, Triton® X-100, polyoxyethylene, PEG 3350, PEG 4000, or a combination thereof.
Aspect 16. The method of Aspect 15, wherein the surfactant is polysorbate 80.
Aspect 17. The method of any one of Aspects 1-16, wherein the surfactant is present in a concentration ranging from 0.001% to 0.5% (w/v) prior to lyophilization.
Aspect 18. The method of any one of Aspects 1-17, wherein the formulation comprises 15 mM glutamate, 9% (wN) sucrose and 0.01% (w/V) polysorbate 80 prior to lyophilization, and wherein the formulation has a pH of 4.2.
Aspect 19. The method of any one of Aspects 1-18, wherein the polypeptide comprises the amino acid sequence of SEQ ID NO: 18 and SEQ ID NO: 9
Aspect 20. The method of any one of Aspects 1-19, wherein the polypeptide comprises the amino acid sequence set forth in SEQ ID NO: 19 or SEQ ID NO: 20.
Aspect 21. The method of any one of Aspects 1-20, wherein the diluent comprises 0.9% benzyl alcohol.
Aspect 22. The method of any one of Aspects 1-20, wherein the diluent comprises phenol.
Aspect 23. The method of any one of Aspects 1-20, wherein the diluent comprises m-Cresol.
Aspect 24. The method of any one of Aspects 1-20, wherein the diluent comprises phenylalanine.
Aspect 25. The method of any one of Aspects 1-24, wherein step (b) comprises administering the reconstituted formulation to a subject in need thereof within six hours of reconstitution.
Aspect 26. The method of any one of Aspects 1-24, wherein step (b) comprises administering the reconstituted formulation to a subject in need thereof within four hours of reconstitution.
Aspect 27. The method of any one of Aspects 1-24, wherein step (b) comprises administering the reconstituted formulation to a subject in need thereof within two hours of reconstitution.
Aspect 28. The method of any one of Aspects 1-24, wherein step (b) comprises administering the reconstituted formulation to a subject in need thereof within one hour of reconstitution.
Aspect 29. The method of any one of Aspects 1-24, wherein the subject is suffering from cancer.
Aspect 30. The method of Aspect 29, wherein the cancer is small cell lung cancer (SCLC), non-small cell lung cancer (NSCLC), glioma, glioblastoma, melanoma, neuroendocrine prostate cancer, neuroendocrine pancreatic cancer, hepatoblastoma, or hepatocellular carcinoma.
Aspect 31. The method of Aspect 29, wherein the cancer is SCLC, NSCLC, glioma, glioblastoma, or neuroendocrine prostate cancer.
Aspect 32. A method comprising (a) reconstituting a lyophilized formulation comprising a polypeptide, a saccharide, a surfactant, and a buffer, with a diluent comprising an aromatic alcohol, a phenolic compound, or an amino acid with aromatic side chain, and (b) administering the reconstituted formulation to a subject in need thereof within 12 hours of reconstitution, wherein the polypeptide is a bispecific antibody construct, such as a bispecific T cell engager.
Aspect 33. The method of Aspect 32, wherein the diluent comprises benzyl alcohol, phenol, m-Cresol, or phenylalanine.
Aspect 34. The method of Aspect 32 or Aspect 33, wherein the pH of the reconstituted formulation is from about 4 to about 7, optionally such as a pH of 4.2.
Aspect 35. The method of any one of Aspects 32-34, wherein the reconstituted formulation comprises about 1 mg/mL to about 20 mg/mL of the polypeptide, such as about 15 mg/mL of the polypeptide.
Aspect 36. The method of any one of Aspects 32-35, wherein the buffer is an acetate buffer, a glutamate buffer, a citrate buffer, a lactic buffer, a succinate buffer, a tartrate buffer, a fumarate buffer, a maleate buffer, a histidine buffer, or a phosphate buffer, optionally a glutamate buffer.
Aspect 37. The method of any one of Aspects 32-36, wherein the buffer is present in at a concentration ranging from 5 to 200 mM prior to lyophilization, such as a concentration ranging from 10 to 50 mM prior to lyophilization.
Aspect 38. The method of any one of Aspects 32-37, wherein the saccharide is a monosaccharide or a disaccharide.
Aspect 39. The method of any one of Aspects 32-38, wherein the saccharide is glucose, galactose, fructose, xylose, sucrose, lactose, maltose, trehalose, sorbitol, mannitol or xylitol, optionally sucrose.
Aspect 40. The method of any one of Aspects 32-39, wherein the saccharide is present in a concentration ranging from 1 to 15% (w/v) prior to lyophilization.
Aspect 41. The method of any one of Aspects 32-40 wherein the surfactant is polysorbate 20, polysorbate 40, polysorbate 60, polysorbate 80, poloxamer 188, poloxamer 407, Triton® X-100, polyoxyethylene, PEG 3350, PEG 4000, or a combination thereof, optionally wherein the surfactant is polysorbate 80.
Aspect 42. The method of any one of Aspects 32-41, wherein the surfactant is present in a concentration ranging from 0.001% to 0.5% (w/v) prior to lyophilization.
Aspect 43. The method of any one of Aspects 32-42, wherein the formulation comprises 15 mM glutamate, 9% (wN) sucrose and 0.01% (wN) polysorbate 80 prior to lyophilization, and wherein the formulation has a pH of 4.2.
Aspect 44. The method of any one of Aspects 32-43, wherein the diluent comprises 0.9% benzyl alcohol, phenol, m-Cresol, and/or phenylalanine.
Aspect 45. The method of any one of Aspects 32-44, wherein step (b) comprises administering the reconstituted formulation to a subject in need thereof within six hours of reconstitution, within four hours of reconstitution, within two hours of reconstitution, or within one hour of reconstitution.
Aspect 46. The method of any one of Aspects 32-45, wherein the subject is suffering from cancer, such as small cell lung cancer (SCLC), non-small cell lung cancer (NSCLC), glioma, glioblastoma, melanoma, neuroendocrine prostate cancer, neuroendocrine pancreatic cancer, hepatoblastoma, or hepatocellular carcinoma.
Aspect 47. The method of any one of Aspects 32-46, wherein the polypeptide comprises any amino acid sequence set forth in the sequence listing provided herewith, such as the amino acid sequence set forth in SEQ ID NO: 20, SEQ ID NO: 21, SEQ ID NO: 22, SEQ ID NO: 23, SEQ ID NO: 33, SEQ ID NO: 43, SEQ ID NO: 44, SEQ ID NO: 45, SEQ ID NO: 55, SEQ ID NO: 65, SEQ ID NO: 66, SEQ ID NO: 76, SEQ ID NO: 77, SEQ ID NO: 87, SEQ ID NO: 97, SEQ ID NO: 98, SEQ ID NO: 99, SEQ ID NO: 109, SEQ ID NO: 110, SEQ ID NO: 111, SEQ ID NO: 121, SEQ ID NO: 122, SEQ ID NO: 131, SEQ ID NO: 141, SEQ ID NO: 142, SEQ ID NO: 146, SEQ ID NO: 147, SEQ ID NO: 156, SEQ ID NO: 165, SEQ ID NO: 174, SEQ ID NO: 183, SEQ ID NO: 184, SEQ ID NO: 185, SEQ ID NO: 186 SEQ ID NO: 187, or SEQ ID NO: 188 (e.g., the amino acid sequence set forth in SEQ ID NO: 76, SEQ ID NO: 77, SEQ ID NO: 97, SEQ ID NO: 98, SEQ ID NO: 99, SEQ ID NO: 156, SEQ ID NO: 165, SEQ ID NO: 174, or SEQ ID NO: 183).

The following example is given merely to illustrate the present invention and not in any way to limit its scope.

### Example

This Example demonstrates a reduction in time required to resolve HMW species upon reconstitution of a lyophilized polypeptide (HLE-BiTE®) formulation using the methods of the disclosure.

A lyophilized HLE-BiTE® formulation (15 mM glutamate, 9% (w/V) sucrose, and 0.01% (wN) polysorbate 80; BiTE® molecule comprising SEQ ID NOs: 75 and 9 (“BiTE®-A”)) was used for the reconstitution study. The diluents used for reconstitution included sterile water for injection (sWFI), WFI comprising 0.9% benzyl alcohol (bWFI), 0.9% saline, saline comprising 0.9% benzyl alcohol, sWFI+0.01% PS80, 5% IVSS (Intravenous Solution Stabilizer)+sWFI, phenylalanine in purified water, phenol in purified water, and m-Cresol in purified water. To conduct the reconstitution, the lyophilized formulation and the reconstitution diluents were first equilibrated at 8° C. or 25° C. for one hour. Then, the proper amount of reconstitution diluent was added to the lyophilized formulation in sterile vials to yield a final protein concentration of 15 mg/mL. The reconstitution time was less than three minutes, during which all the lyophilized cake completely dissolved and the solution was gently swirled for sample homogeneity. The reconstituted samples were immediately transferred to an ultra-performance liquid chromatography (UPLC) sample manager with a preset 8° C. or 25° C. The estimated time from reconstitution to the first injection of size exclusion-ultra-performance liquid chromatography (SE-UHPLC) time point was less than five minutes. For each SE-UHPLC injection, 10 μg of sample was loaded onto a BEH200 UPLC column (1.7 μm 4.6×150 mm) connected to an ACQUITY UPLC system (Waters, Milford, MA). The mobile phase was 100 mM sodium phosphate with 250 mM sodium chloride at pH 6.8 at a flow rate of 0.4 mL/min. The UPLC sample manager was held at either 8° C. or 25° C. The results were analyzed using Chromeleon® software, and the peak integration was calculated using the UV absorbance at 220 nm. The pH and concentration of reconstituted diluent were measured afterwards with standard pH probe (Mettler Toledo, OH) and UV absorbance at 280 nm using an extinction coefficient of 1.8.

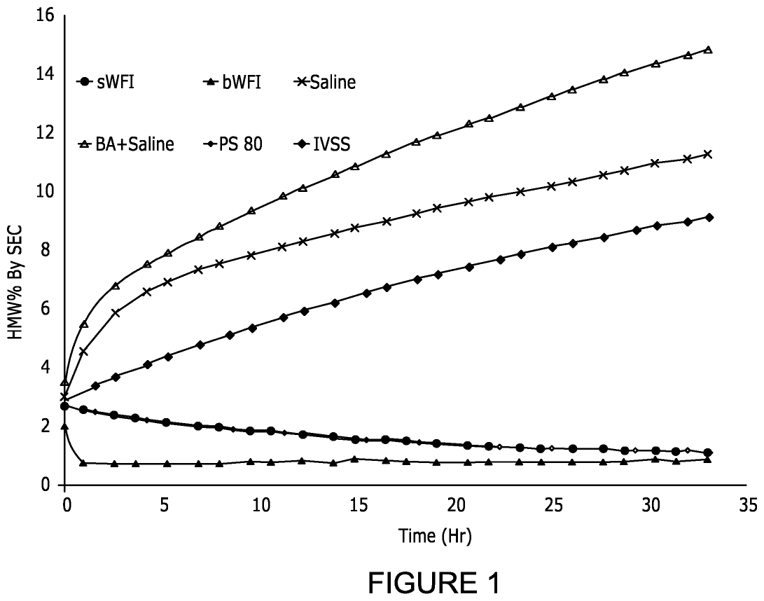

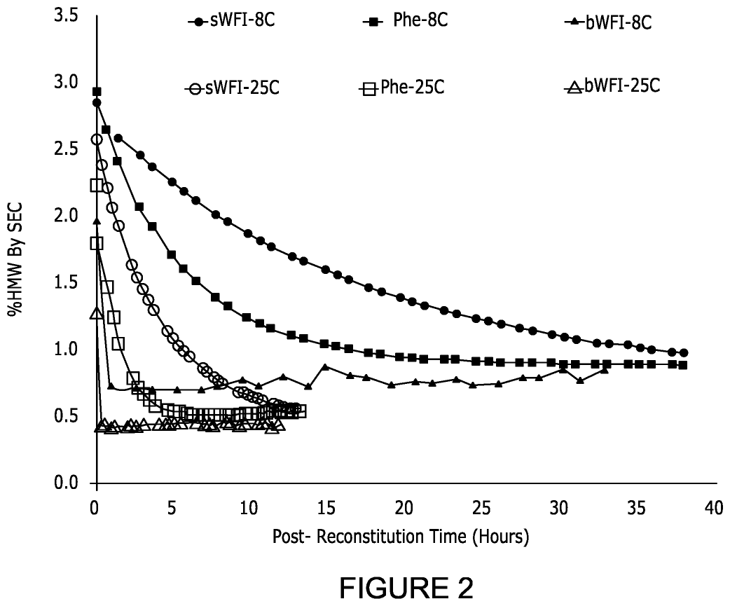

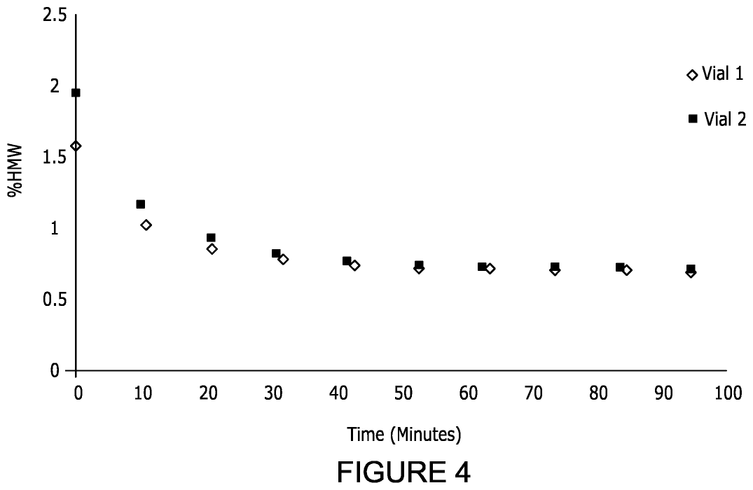

FIG. 1 illustrates a reconstitution study performed at 8° C. Over 24 hours was required for the reconstituted formulation using sWFI and polysorbate 80 as diluent to reach “equilibrium” (i.e., a consistent level of % of HMW species). Saline, IVSS, and saline+benzyl alcohol significantly increased the percentage of HMW in the reconstituted formulation, and HMW levels continued to climb throughout the study. Remarkably, use of bWFI as a diluent resulted in equilibration within one hour of reconstitution. FIG. 2 provides the results of a similar study conducted at 25° C. using sWFI and bWFI, comparing the time to equilibrium at 8° C. and at 25° C. HMW species dissociated more quickly at 25° C. for both sWFI and bWFI. At both temperatures, bWFI significantly accelerated the dissociation process compared to sWFI, with equilibrium achieved in less than an hour at 25° C. bWFI resulted in reconstitution formulations comprising less than 1% HMW species. Indeed, at 25° C., the percentage of HMW species was reduced to about 1% or less within 20 minutes of reconstitution and remained at that level for at least 100 minutes. See FIG. 4.

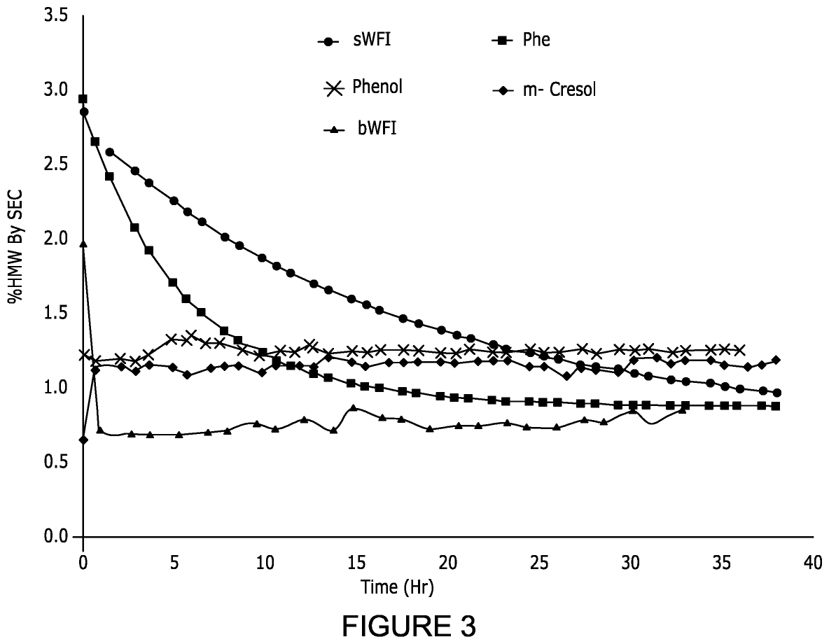

Additional diluents were studied providing the results illustrated in FIG. 3. FIG. 3 illustrates data collected during the study at 8° C. Phenylalanine, phenol, and m-cresol accelerated HMW species dissociation compared to sWFI. Use of all diluents tested other than sWFI resulted in a reconstituted formulation with less than 1.5% HMW species by 10 hours post-reconstitution, with bWFI-based reconstitution formulations demonstrated the lowest levels of HMW species.

Additional studies were performed using materials and methods similar to those set forth above. Four lyophilized BITEs in vials were used with reconstitution media including sterile water for injection (sWFI), bacteriostatic WFI (bWFI), 0.9% saline, and phenol. BiTE®-A comprised SEQ ID NOs: 75 and 9 (including CDR sequences SEQ ID NOs: 67-72 and 1-6); BiTE®-B comprised SEQ ID NO: 156 (including CDR sequences SEQ ID NOs: 148-153 and 1-6); BiTE®-C comprised SEQ ID NO: 97 (including CDR sequences SEQ ID NOs: 88-93 and 1-6); and BiTE®-D comprised SEQ ID NO: 98 (including CDR sequences SEQ ID NOs: 88-93 and 1-6). Additionally, for BiTE®-A (BiTE® molecule comprising SEQ ID NOs: 75 and 9), studies were performed using bacteriostatic saline, sWFI+0.01% PS80, 5% IVSS (Intravenous Solution Stabilizer)+sWFI, phenylalanine, and m-cresol. For reconstitution, the lyophilized BiTE vials and all the reconstitution solutions were first equilibrated at specified temperatures, 25° C. or 8° C., for 1 hour. Then, the appropriate volume of reconstitution solution was added to the lyophilized vials. Samples were reconstituted, up to three minutes, until the lyophilized cake was completely dissolved. The sample was then gently swirled for sample homogeneity. The reconstituted samples were then transferred immediately to a UPLC sample manager preset to 8° C. or 25° C. The estimated time from reconstitution to the first injection on SE-UHPLC was <5 minutes. For SE-UPLC, the appropriate amount of the sample was loaded onto a SEC column connected to an ACQUITY UPLC system (Waters, Milford, MA). The UPLC sample manager was held at either 8° C. or 25° C. The results were analyzed using Chromeleon® software and the peak integration was calculated using the UV absorbance or fluorescence detection. The pH and concentration of reconstituted samples were measured afterwards with a standard pH probe (Mettler Toledo, OH) and UV absorbance at 280 nm.

To compare the effect of reconstitution media on different BiTEs, t_{90%} is defined as the time duration needed to have 90% of the initial high molecular weight species (HMW) dissociated back to monomer; t_{90%} is calculated as follows:

$$\frac{d [ HMW ]}{dt} = k [ HMW ]$$

$$[ HMW ] = {[ HMW ]}_{t 0} {e}^{- kt}$$

$${t}_{90 \%} = - \frac{ln ( 0.1 )}{k}$$

[HMW] is the concentration of the high molecular weight species, [HMW] to is the concentration of the high molecular weight species at time zero, a first order equation was fitted to the dissociation curve and k is the rate constant.

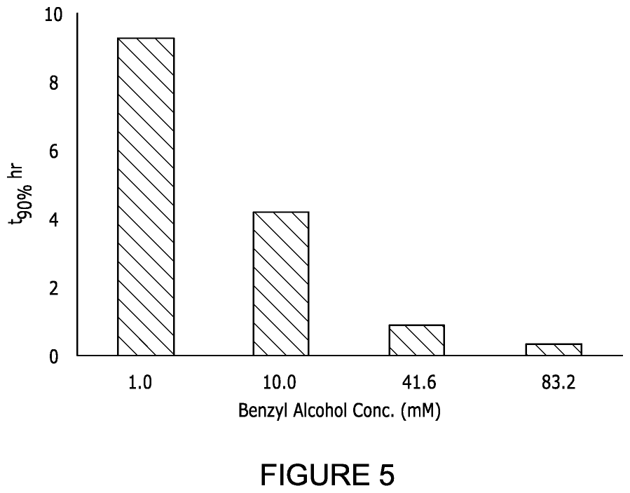

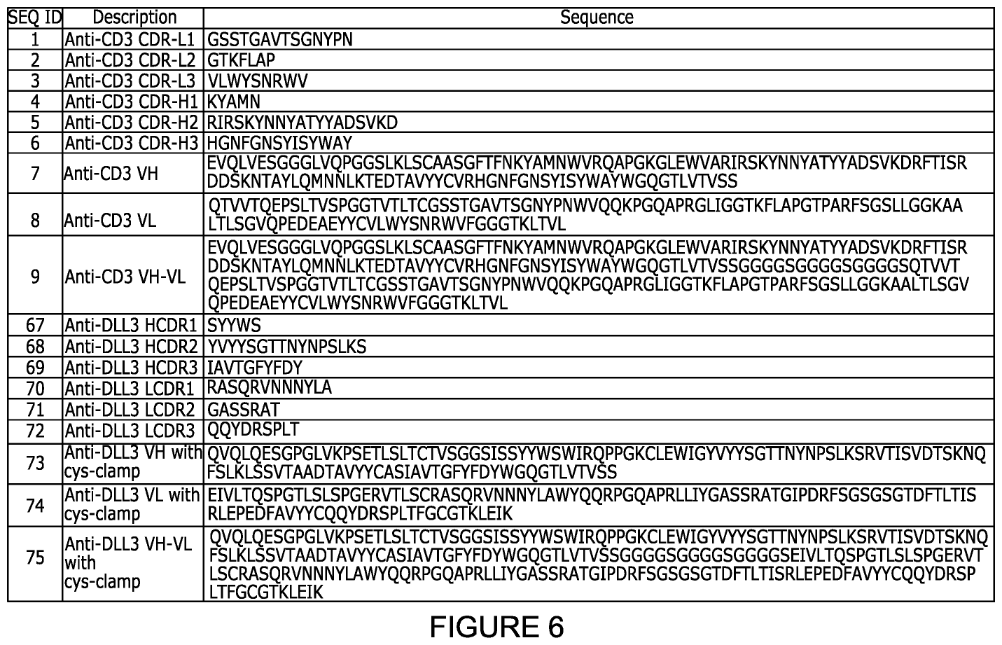

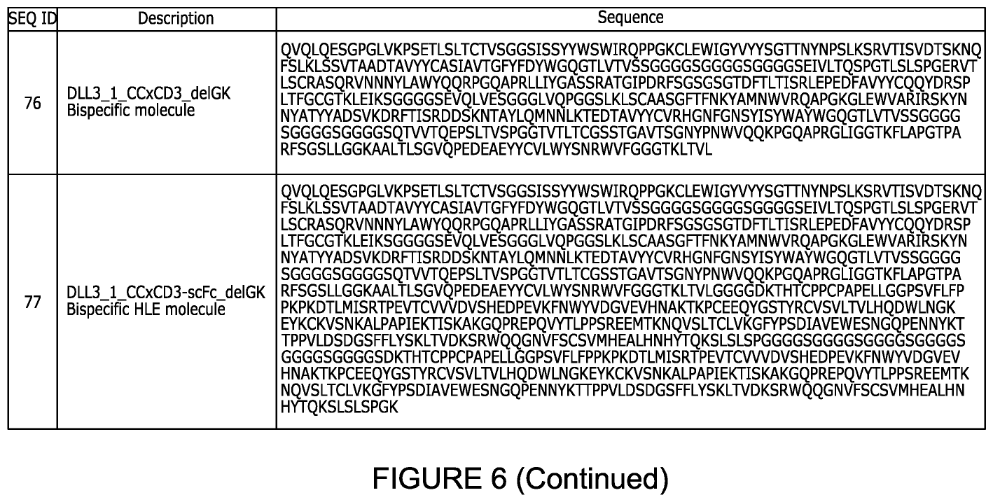

The results of the studies are presented in FIG. 5 and Table 1. FIG. 5 illustrates the effects of benzyl alcohol concentration on the dissociation of % HMW of BiTE®-A reconstituted as 15 mg/mL at 25° C., as indicated by t_{90%} as defined above. Dissociation of HMW species was improved at all concentrations of benzyl alcohol, and concentrations greater than 40 mM (e.g., 41.6 mM and 83.2 mM) led to a “reconstitution duration time” (i.e., a time following reconstitution wherein 90% of the initial high molecular weight species (HMW) formed dissociate back to monomer) of less than two hours. The data show that a reconstitution duration time of less than 1 hour may be achieved with benzyl alcohol concentrations higher than 41.6 mM (e.g., higher than 42 mM).

Table 1 below summarizes results from the studies demonstrating the effects of the tested reconstitution media on the dissociation time of various BiTEs® at 25° C., as indicated by t_{90%} as defined above. For all samples tested, both benzyl alcohol and phenol greatly shortened the reconstitution duration period as compared to WFI, typically less than 1 hour vs. 9+hours. For BiTE®-B and BiTE®-C, less than 1 hour is reported due to the fast kinetics. In contrast to other conditions, saline promoted aggregation (Agg.) for BiTE®-A, BiTE®-B and BiTE®-C, and extended t_{90%} for BiTE®-D from 19.1 hours to 26.1 hours.

TABLE 1

| | WFI | Benzyl Alcohol | Phenol | Saline |
|---|---|---|---|---|
| | (hours) | (hours) | (hours) | (hours) |
| BiTE ®-A | 9.3 | 0.3 | 0.5 | Agg. |
| BiTE ®-B | 9.0 | <1.0 | <1.0 | Agg. |
| BiTE ®-C | 10.0 | <1.0 | <1.0 | Agg. |
| BiTE ®-D | 19.1 | 1.0 | 1.1 | 26.1 |

The Example demonstrates that reconstitution of the lyophilized polypeptide formulation (e.g., an HLE-BiTE® product formulation) described herein with a diluent comprising WFI comprising 0.9% benzyl alcohol, phenylalanine, phenol, or m-cresol significantly decreased the time required for dissociation of HMW species compared to other diluents tested, including WFI alone. Reconstituted formulations comprising less than 1.5% HMW species was achieved within 15 hours of reconstitution at 8° C. using these diluents. A diluent comprising 0.9% benzyl alcohol (bWFI) outperformed the other diluents tested in both reducing the time required to dissociate HMW species (i.e., accelerating the time to equilibrium) following reconstitution and achieving a final reconstituted formulation with reduced HMW species content. The data reported herein further establishes that use of a diluent comprising an aromatic alcohol, a phenolic compound, or an amino acid with aromatic side chain achieves desirable reconstitution duration times, including less than 1.5 hours. The reconstitution duration times were significantly reduced compared to reconstitution using water for injection or saline for all constructs tested.

While this invention has been described with an emphasis upon preferred aspects, it will be obvious to those of ordinary skill in the art that variations of the preferred compounds and methods may be used and that it is intended that the invention may be practiced otherwise than as specifically described herein. Accordingly, this invention includes all modifications encompassed within the spirit and scope of the invention as defined by the following claims.

What is claimed is:

1. A method comprising
(a) reconstituting a lyophilized formulation comprising a polypeptide, a saccharide, a surfactant, and a buffer, with a diluent comprising an aromatic alcohol, a phenolic compound, or an amino acid with aromatic side chain, and
(b) administering the reconstituted formulation to a subject in need thereof within 12 hours of reconstitution,
wherein the polypeptide is a bispecific antibody construct.

2. The method of claim 1, wherein the diluent comprises benzyl alcohol, phenol, m-Cresol, or phenylalanine.

3. The method of claim 1 or claim 2, wherein the pH of the reconstituted formulation is from about 4 to about 7.

4. The method of any one of claims 1-3, wherein the formulation has a pH of 4.2.

5. The method of any one of claims 1-4, wherein the reconstituted formulation comprises about 1 mg/mL to about 20 mg/mL of the polypeptide.

6. The method of any one of claims 1-5, wherein the reconstituted formulation comprises about 15 mg/mL of the polypeptide.

7. The method of any one of claims 1-6, wherein the buffer is an acetate buffer, a glutamate buffer, a citrate buffer, a lactic buffer, a succinate buffer, a tartrate buffer, a fumarate buffer, a maleate buffer, a histidine buffer, or a phosphate buffer.

8. The method of claim 7, wherein the buffer is a glutamate buffer.

9. The method of any one of claims 1-8, wherein the buffer is present in at a concentration ranging from 5 to 200 mM prior to lyophilization.

10. The method of claim 9, wherein the buffer is present at a concentration ranging from 10 to 50 mM prior to lyophilization.

11. The method of any one of claims 1-10, wherein the saccharide is a monosaccharide or a disaccharide.

12. The method of any one of claims 1-11, wherein the saccharide is glucose, galactose, fructose, xylose, sucrose, lactose, maltose, trehalose, sorbitol, mannitol or xylitol.

13. The method of claim 12, wherein the saccharide is sucrose.

14. The method of any one of claims 1-13, wherein the saccharide is present in a concentration ranging from 1 to 15% (w/v) prior to lyophilization.

15. The method of any one of claims 1-14 wherein the surfactant is polysorbate 20, polysorbate 40, polysorbate 60, polysorbate 80, poloxamer 188, poloxamer 407, Triton® X-100, polyoxyethylene, PEG 3350, PEG 4000, or a combination thereof.

16. The method of claim 15, wherein the surfactant is polysorbate 80.

17. The method of any one of claims 1-16, wherein the surfactant is present in a concentration ranging from 0.001% to 0.5% (w/v) prior to lyophilization.

18. The method of any one of claims 1-17, wherein the formulation comprises 15 mM glutamate, 9% (wN) sucrose and 0.01% (w/V) polysorbate 80 prior to lyophilization, and wherein the formulation has a pH of 4.2.

19. The method of any one of claims 1-18, wherein the diluent comprises 0.9% benzyl alcohol.

20. The method of any one of claims 1-18, wherein the diluent comprises phenol.

21. The method of any one of claims 1-18, wherein the diluent comprises m-Cresol.

22. The method of any one of claims 1-18, wherein the diluent comprises phenylalanine.

23. The method of any one of claims 1-22, wherein step (b) comprises administering the reconstituted formulation to a subject in need thereof within six hours of reconstitution.

24. The method of any one of claims 1-22, wherein step (b) comprises administering the reconstituted formulation to a subject in need thereof within four hours of reconstitution.

25. The method of any one of claims 1-22, wherein step (b) comprises administering the reconstituted formulation to a subject in need thereof within two hours of reconstitution.

26. The method of any one of claims 1-22, wherein step (b) comprises administering the reconstituted formulation to a subject in need thereof within one hour of reconstitution.

27. The method of any one of claims 1-22, wherein the subject is suffering from cancer.

28. The method of any one of claims 1-27, wherein polypeptide binds DLL3 and CD3.

29. The method of any one of claims 1-28, wherein the polypeptide comprises the amino acid sequence of SEQ ID NO: 75 and SEQ ID NO: 9

30. The method of any one of claims 1-29, wherein the polypeptide comprises the amino acid sequence set forth in SEQ ID NO: 76 or SEQ ID NO: 77.

31. The method of any one of claims claim 27-30, wherein the cancer is small cell lung cancer (SCLC), non-small cell lung cancer (NSCLC), glioma, glioblastoma, melanoma, neuroendocrine prostate cancer, neuroendocrine pancreatic cancer, hepatoblastoma, or hepatocellular carcinoma.

32. The method of claim 31, wherein the cancer is SCLC, NSCLC, glioma, glioblastoma, or neuroendocrine prostate cancer.

33. The method of any one of claims 1-27, wherein the polypeptide comprises the amino acid sequence set forth in SEQ ID NO: 20, SEQ ID NO: 21, SEQ ID NO: 22, SEQ ID NO: 23, SEQ ID NO: 33, SEQ ID NO: 43, SEQ ID NO: 44, SEQ ID NO: 45, SEQ ID NO: 55, SEQ ID NO: 65, SEQ ID NO: 66, SEQ ID NO: 76, SEQ ID NO: 77, SEQ ID NO: 87, SEQ ID NO: 97, SEQ ID NO: 98, SEQ ID NO: 99, SEQ ID NO: 109, SEQ ID NO: 110, SEQ ID NO: 111, SEQ ID NO: 121, SEQ ID NO: 122, SEQ ID NO: 131, SEQ ID NO: 141, SEQ ID NO: 142, SEQ ID NO: 146, SEQ ID NO: 147, SEQ ID NO: 156, SEQ ID NO: 165, SEQ ID NO: 174, SEQ ID NO: 183, SEQ ID NO: 184, SEQ ID NO: 185, SEQ ID NO: 186, SEQ ID NO: 187, or SEQ ID NO: 188.

34. The method of claim 33, wherein the polypeptide comprises the amino acid sequence set forth in SEQ ID NO: 76, SEQ ID NO: 77, SEQ ID NO: 97, SEQ ID NO: 98, SEQ ID NO: 99, SEQ ID NO: 156, SEQ ID NO: 165, SEQ ID NO: 174, or SEQ ID NO: 183.
